SEQUENCE LISTING (18 sequences)

SEQ ID NO: 1 - DNA, 1090 bp, Homo sapiens
  CDS (212)..(778)
ccccagccagctgtcccgagtctggactttccctctgcccctccccactctcaggctggtggggtggggaaagcagcccattcctgggctcagagactcccaccccagctcagagggagcaggggcccagccagggacggaccctcattcctcccagggaccccagacctctgtctctctcgggccttggctccttgaacttttggccgccatgtgcttcccgaaggtcctctctgatgacatgaagaagctgaaggcccgaatgcaccaggccatagaaagattttatgataaaatgcaaaatgcagaatcaggacgtggacaggtgatgtcgagcctggcagagctggaggacgacttcaaagagggctacctggagacagtggcggcttattatgaggagcagcacccagagctcactcctctacttgaaaaagaaagagatggattacggtgccgaggcaacagatcccctgtcccggatgttgaggatcccgcaaccgaggagcctggggagagcttttgtgacaaggtcatgagatggttccaggccatgctgcagcggctgcagacctggtggcacggggttctggcctgggtgaaggagaaggtggtggccctggtccatgcagtgcaggccctctggaaacagttccagagtttctgctgctctctgtcagagctcttcatgtcctctttccagtcctacggagccccacggggggacaaggaggagctgacaccccagaagtgctctgaaccccaatcctcaaaatgaagatactgacaccacctttgccctccccgtcaccgcgcacccaccctgacccctccctcagctgtcctgtgccccgccctctcccgcacactcagtccccctgcctggcgttcctgccgcagctctgacctggtgctgtcgccctggcatcttaataaaacctgcttatacttccctggcaggggagataccatgatcgcggaggtgggtttcccagggcaaggctgatctgttgccgtattagtccgttttcacacagctataaagaatgcctgagactgggtgatgtataaagaaaagaagtttaactga

SEQ ID NO: 2 - protein, 188 aa, Homo sapiens
MCFPKVLSDDMKKLKARMHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 3 - DNA, 26 bp, Homo sapiens
gggaattcatgtgcttcccgaaggtc

SEQ ID NO: 4 - DNA, 26 bp, Homo sapiens
ggctcgagtcattttgaggattgggg

SEQ ID NO: 5 - DNA, 26 bp, Homo sapiens
atgtgcttcccgaaggtcctctctga

SEQ ID NO: 6 - DNA, 26 bp, Homo sapiens
tcattttgaggattggggttcagagc

SEQ ID NO: 7 - DNA, 20 bp, Homo sapiens
ccacagtcacctatggcaac

SEQ ID NO: 8 - DNA, 20 bp, Homo sapiens
agtgtctcctggctctggtt

SEQ ID NO: 9 - DNA, 20 bp, Homo sapiens
gcttcaggagctgaataccc

SEQ ID NO: 10 - DNA, 20 bp, Homo sapiens
aaggatcacgaccatcttcc

SEQ ID NO: 11 - DNA, 20 bp, Homo sapiens
tgaacccaacaataggcaaa

SEQ ID NO: 12 - DNA, 20 bp, Homo sapiens
cctctcatcattccacatgc

SEQ ID NO: 13 - DNA, 20 bp, Homo sapiens
cccaactggtacatcagcac

SEQ ID NO: 14 - DNA, 20 bp, Homo sapiens
ggaagacacaaattgcatgg

SEQ ID NO: 15 - DNA, 20 bp, Homo sapiens
cctcttctccttcctgatcg

SEQ ID NO: 16 - DNA, 20 bp, Homo sapiens
atcactccaaagtgcagcag

SEQ ID NO: 17 - DNA, 20 bp, Homo sapiens
cgacttcaaagagggctacc

SEQ ID NO: 18 - DNA, 19 bp, Homo sapiens
gagtgagctctgggtgctg

(19) United States
(12) Patent Application Publication
(10) Pub. No.: US 2009/0263377 A1
(43) Pub. Date: Oct. 22, 2009

(54) IL-32B-TARGETED DIAGNOSIS AND THERAPY

(76) Inventor: Charles Lin, Brentwood, TN (US)

Correspondence Address: FULBRIGHT & JAWORSKI L.L.P., 600 CONGRESS AVE., SUITE 2400, AUSTIN, TX, 78701, US

(21) Appl. No.: 12/420,972

(22) Filed: Apr. 9, 2009

Related U.S. Application Data
(60) Provisional application No. 61/043,954, filed on Apr. 10, 2008.

(51) Int. Cl. A61K 39/395; C12Q 1/02; C12Q 1/68; A61K 38/16; A61K 31/7088; A61P 35/00

(52) U.S. Cl. 424/130.1; 435/29; 435/6; 514/12; 514/44 A

(57) ABSTRACT

Methods for the diagnosis and treatment of IL-32β over-expressing cancers are provided. Also provided are methods for the treatment of wounds, hair loss and the promoting and/or restoration of hair growth utilized IL-32β antagonists. Finally, a sepsis model utilizing IL-32β is provided, as are methods for treating sepsis and inflammation using IL-32β antagonists.

The present application claims benefit of priority to U.S. Provisional Application Ser. No. 61/043,954, filed Apr. 10, 2008, the entire contents of which are hereby incorporated by reference.

This invention was made with government support under grant nos. R01-NS45888, R01-CA108856, and R01-AR053718 awarded by the National Institutes of Health. The government has certain rights in the invention.

### BACKGROUND OF THE INVENTION

I. Field of the Invention

The present invention relates to the fields of oncology, genetics and molecular biology. More particular the invention relates to the elucidation of a key role for IL-32β in inflammation and sepsis. In addition, the invention relates to a role for IL-32β in cancer, particularly in a diagnostic context, and in hair growth.

II. Related Art

Inflammation is a protective reaction elicited by the host in response to infection, injury, and tissue damage. Infiltration of lymphocytes, macrophages, mast cells and neutrophils is a hallmark of inflammatory defenses and tissue repair reactions. Inflammation has been directly linked to a variety of disease development and progression.

Vascular endothelium is an active participant in inflammation. Inflamed endothelium plays diverse activities that include the regulation of leukocyte recruitment and infiltration, cytokine production, protease and extracellular matrix synthesis, and vascular permeability (Folkman, 1995; Folkman, 2001). For inflammation to occur, circulating leukocytes must first be able to adhere selectively and efficiently to microvascular endothelium in order to withstand the shear force exerted by the flowing blood.

Adhesion of leukocytes to the activated endothelium is facilitated by induction of vascular cell adhesion molecules on the inflamed endothelium, such as vascular cell adhesion molecule (VCAM)-1, intercellular adhesion molecules (ICAM)-1, and E-selectin, which subsequently leads to transendothelial migration to disease sites. It is evident that the endothelium is a critical gatekeeper that controls the recruitment of distinct leukocyte subpopulations and, in doing so, determines the nature and extent of acute and chronic inflammation. Endothelium also exerts a potent inflammatory role by secreting inflammatory cytokines such as IL-1 and TNFα, which upregulates ICAM-1 expression and enhances leukocyte adherence to the activated endothelium.

The phosphatidylinositol-3 kinase (PI3K)-Akt signaling axis regulates angiogenesis, vascular survival and homeostasis (Shiojima and Walsh, 2002). In searching for Akt activated genes in endothelium, the inventor identified natural killer cell transcript 4 (NK4) was dramatically induced by Akt activation in cultured human endothelial cells. NK4 was originally isolated from activated human natural killer cells in 1992 (Dahl et al., 1992). The expression of NK4 is increased upon activation of T cells by mitogens or activation of NK cells by IL-2. No homology was found between the sequence of the coding region of NK4 and any sequences in the GenBank data base (Dahl et al., 1992).

Recently, this gene was rediscovered in human lymphocytes upon IL-18 stimulation, and renamed as IL-32 (Kim et al., 2005). Although IL-32 does not share sequence homology with any known cytokine families, IL-32 induces various cytokines, such as TNFα and IL-8 expression in monocytic cells. IL-32 activates the NF-κB and p38 mitogen-activated protein kinase in those lymphocytes. The full length IL-32 gene consists of 705 base pair. Sequence analysis predicted a molecular weight of 27 kDa. Human IL-32 exists as four splice variants in blood cells, named IL-32α, β, γ and δ, with IL-32α as the major isoform (Kim et al., 2005). The highest homology to human IL-32 was found in bovine tissue at 31.8%, and no homologue to this gene was found in mice. Since IL-32 expression is regulated by inflammatory cytokines in human peripheral lymphocyte cells, it has been speculated that it may play a role in inflammatory/autoimmune diseases (Kim et al., 2005). Further analysis indeed showed a elevation of IL32 in human inflammatory diseases, such as in rheumatoid arthritis (Shoda et al., 2006; Joosten et al., 2006; Cagnard et al., 2005), ulcerative colitis and Crohn's disease (Netea et al., 2005; Shioya et al., 2007).

### SUMMARY OF THE INVENTION

In accordance with the present invention, there is provided a method of diagnosing cancer comprising (a) obtaining a cell sample from a subject suspected of having or at risk of developing cancer; (b) assessing IL-32β expression in one or more cells of said sample; and (c) comparing IL-32β expression to that observed in one or more normal cells; wherein elevated IL-32β expression in the cell or cells of step (b), as compared to the cell or cells of step (c), indicates that said subject has cancer. The cancer, in particular, may be breast cancer. The sample may be blood or serum, and/or may comprise endothelial cells. Assessing may comprise detection of protein levels, such as by ELISA, RIA, western blot, immunohistochemistry, for FACS. Assessing may comprise detection of mRNA levels, such as by Northern blot or quantitative RT-PCT. The method may further comprise assessing IL-32β expression in one or more normal cells. The method may also further comprise making a treatment decision based on the result in step (c).

In another embodiment, there is provided a method of treating cancer in a subject, said cancer being characterized by overexpression of IL-32β, comprising administering to said subject an antagonist of IL-32β function or expression. The cancer may be breast cancer. The cancer may be recurrent, multi-drug resistant and/or metastatic. The antagonist may be an antibody to IL-32β, or a peptide or polypeptide that blocks IL-32β to its receptor. The antagonist also may be an inhibitory RNA, a small molecule. The antagonist may be administered topically, orally, intravenously, intra-arterially, subcutaneously, intradermally, or intratumorally. The method may further comprise administering to said subject a second cancer therapy, such as chemotherapy, radiotherapy, immunotherapy, hormone therapy, toxin therapy, or surgery. Multiple administration of the IL-32β antagonist and/or other agent are contemplated.

In yet another embodiment, there is provided a method or promoting or restoring hair growth in a subject comprising administering to a hair-producing cell of said subject an antagonist of IL-32β function or expression. The subject may be a human subject. The antagonist is an antibody to IL-32β or a peptide or polypeptide that blocks IL-32β to its receptor. The antagonist also may be an inhibitory RNA or a small molecule. The antagonist may be administered orally, intravenously, intra-arterially, subcutaneously, intradermally or topically. The method may further comprise administering to said subject a second hair growth promoting or restoring agent. Multiple administration of the IL-32β antagonist and/or other agent are contemplated.

In still a further embodiment, there is provided a method for preparing an animal model for sepsis comprising (a) providing a mouse; and (b) introducing into the vasculature of said mouse transgenic murine endothelial cells that overexpress IL-32β as compared to normal murine endothelial cells. Yet an additional embodiment comprises a method of treating sepsis in a subject comprising administering to said subject an IL-32β-specific antagonist.

In another embodiment, there is provided a method promoting wound healing in a subject comprising administering to said subject an antagonist of IL-32β function or expression. The subject is may be a human subject. The antagonist may be an antibody to IL-32β, a peptide or polypeptide that blocks IL-32β to its receptor, an inhibitory RNA or a small molecule. The antagonist may be administered orally, intravenously, intra-arterially, subcutaneously, intradermally or topically, or directly to a wound site. The method may further comprise administering to said subject a second wound healing agent. Also provided is a wound covering or bandage comprising an antagonist of IL-32β expression or function. The covering/dressing may be a bandage, patch, pad gauze, or mesh. Multiple administration of the IL-32β antagonist and/or other agent are contemplated.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIGS. 1A-C: Characterization and cloning of IL-32 (NK4). HUVECs were infected with adenoviral vectors directing the expression of Akt (lane 3) or control vector (AdGFP, lane 2), respectively, for 48 hours (MOI 10). Non-infected cells were used as a control (lane 1). IL-32 (NK4) mRNA was analyzed by Northern blot. 18S rRNA, visualized with ethidium bromide and UV, was used as a loading control (FIG. 1A). Images are representative of 3 separate experiments. IL-32 was amplified by RT-PCR from Akt transfected HUVECs and cloned. Sequence analysis indicates there exists multiple splicing isoforms, IL-32α, IL-32β, and IL-32ε in addition to the full length gene, IL-32γ (FIG. 1B). Protein sequences alignment of each IL-32 isoforms (FIG. 1C).

FIGS. 2A-B: IL-32 is expressed only in human tissues and predominantly in endothelial cells. IL-32 was amplified using a human tissue cDNA panel by PCR (FIG. 2A). G3PDH was used as an internal control. Total RNAs were isolated from different types of human cells and analyzed by Northern blot for IL-32 mRNA. 18S & 28S rRNA, visualized with ethidium bromide and UV, were used as a loading control (FIG. 2B). The experiments were repeated more than twice.

FIG. 3: IL-32 is expressed in certain primates. Southern blot was performed on DNA isolated from mouse (lane 1), ring-tailed lemur (lane 2), common woolly monkey (lane 3), black-handed spider monkey (lane 4), red-chested mustached tamari (lane 5), pigtailed macaque (lane 6), rhesus monkey (lane 7), Sumatran orangutan (lane 8), gorilla (lane 9), chimpanzee (lane 10), bonobo (lane 11), HUVECs (lane 12). Genomic DNA, visualized with ethidium bromide and UV, were used as a loading control. The experiments were repeated at least 2 times.

FIGS. 4A-D: IL-32β is an intracellular protein. HUVECs were infected with either Ad-IL32β, direct the expression of IL-32β fused with V5 and 6His epitope tags at the C-terminus of the gene, or control vector (AdGFP) for 48 hours. Cell lysate and concentrated culture media (7-fold concentration) were analyzed for IL-32β protein by ELISA (FIG. 4A), and Western blot (FIG. 4B) using anti V5 and 6His-tag specific antibodies. For western blot, cell lysate and conditioned medium were immunoprecipitated with an anti V5 antibody, subsequently probed with an anti-V5 antibody to detect IL-32β protein. β-tubulin was used as a control for equal loading in cell lysate. Ponceau S staining was used to confirm equal loading for the conditioned medium (data not shown). GFP tagged IL-32β was transfected into HUVECs. Cellular localization of IL-32β was imaged in live cells 48 hr after transfection (FIG. 4C). BAVECs were transfected with either an EGFP-IL-32β expression vector or a control EGFP expression vector respectively, followed by incubation with ER-Tracker™ Red. Cellular IL-32β (green) and ER (red) in endothelial cells was imaged by Confocal microscopy (FIG. 4D). The biodistribution of both markers was analyzed and graphed by optical cross sections (red lines) (FIG. 4D). The experiments were repeated at least 3 times.

FIGS. 5A-C: IL-1β or TNFα induce IL-32 expression independently of Akt and dependently of NF-κB. HUVECs were infected with control Adβ-gal, AdDN-Akt, AdIκB, and AdAkt plus AdIκB adenovirus (MOI=10) for 36 hr. Uninfected cells were used as a control (none), followed by incubation with recombinant IL-1β (FIG. 5A) or TNFα (FIG. 5B) at 10 ng/ml for 12 hr. IL-32 mRNA was analyzed by Northern blot. 18S rRNA, visualized with ethidium bromide and UV, was used as a loading control. Akt kinase activity was evaluated by the GSK3α/β fusion protein phosphorylation assay in cells treated in each group (FIG. 5C). The experiments were repeated at least 3 times.

FIGS. 6A-E: IL-32β induces the expression of vascular cell adhesion molecule and leukocyte adhesion on activated endothelial cells. HUVECs were infected with either AdIL-32β or AdGFP for 48 hours, and then treated with IL-1β at 100 pg/ml for 4 hr. qRT-PCR was performed for the indicated adhesion molecules. “-fold” change of gene expression was calculated against the GFP control (FIGS. 6A-C). HUVECs were infected with either control AdGFP or AdIL-32β, for 36 hr followed by incubation with IL-1β at the dose indicated for 4 hours. THP-1 leukocyte adhesion was measured 30 min after incubation (FIG. 6D). ECs isolated from WT or PECAM-IL32 mice were used for THP-1 adhesion assay as described for panel D (FIG. 6E). The experiments were repeated at least 3 times. The data were presented as mean±SEM. *p<0.05.

FIGS. 7A-B: IL-32β sensitizes proinflammatory cytokine production. HUVECs were infected with either AdGFP or AdIL-32β for 24 hr, followed by incubation without or with IL-1β at 100 pg/ml for the indicated amount of time. IL-1β expression was analyzed by real time PCR (FIG. 7A). Diagram of positive feedback of IL-32 in vascular inflammation (FIG. 7B).

FIGS. 8A-B: IL-32β exacerbates inflammation in sepsis. WT and PECAM-IL32 mice underwent CLP surgery. Survival curve. n=8 for each group (FIG. 8A). Serum TNFα and IL-1β were measured at 17 hr following CLP surgery. The data were presented as mean±SEM. *p<0.05. n>3 for each group (FIG. 8B).

FIG. 9: Endothelium is a major source of chemokines, growth factors and adhesion molecules that contributes to disease development.

FIG. 10: Real-time RT-PCR of IL-32β mRNA on human breast cancer (BC) samples versus adjacent non-cancerous tissue (Normal). The level of IL-32 in normal tissues was set as 1, and the fold of increase in tumor vs normal was calculated in each paired samples accordingly. Fourteen paired samples from fourteen patients were analyzed.

FIG. 11: IL-32 expression is elevated in brain tumor tissues, and its expression correlates with tumor malignancy. Grade 1 (G1), 2 (G2) and 4 (G4) brain tumor tissues were collected from patients at Vanderbilt Hospital. Control (con) sample was collected from patient with epilepsy. Total RNAs were isolated from fresh tissues and subjected to semi quantitative RT-PCR using specific primer set for IL-32. β-actin was used as a internal control.

FIG. 12: Expression of IL-32β in mice accelerate breast cancer development. MMTV-neu transgenic mice were crossed with IL-32β transgenic mice (PECAM-IL32). Mammary tumor development was examined weekly starting 23 weeks after birth. Percentage of mice that carry tumors were calculated and graphed. n=30 per group.

FIG. 13: IL-32β is predominantly expressed in human endothelial cells. Northern blot analysis of IL-32 expression in cellular RNA collected from different cell lines: DU145, HEK293 and MCF7 are epithelial cells; HL60 is neutrophils; THP-1 is monocytes; CCD21-SK is fibroblasts; HASMC is smooth muscle cell; HCAEC and HUVEC are endothelial cells. 18S & 28S rRNAs were used as a loading control.

FIG. 14: Southern blot for IL-32 DNA. Performed on evolutionary primate genomic DNA panel (purchased from Emory Primate Center). Evolution time scale of each species is listed.

FIG. 15: HUVECs were transduced with adenoviral vector expressing GPF as a viral control, dominant negative Akt (DN-Akt), or mutant IκB for 24 hr. The cells were then stimulated with IL-1β at 10 ng/ml. Unstimulated cells (none) were used as a control. Northern blot was performed for IL-32β expression 8 hr after IL-1β stimulation (top panel). rRNA was used as a loading control (bottom panel).

FIGS. 16A-B: IL-32β induces the expression of vascular cell adhesion molecule and leukocyte adhesion on activated endothelial cells. HUVECs were infected with either AdIL-32β or AdGFP for 48 hr, and then treated with IL-1β at 100 pg/ml for 4 hr. qRT-PCR was performed for the indicated adhesion molecules. “-fold” change of gene expression was calculated against the GFP control (FIG. 16A). HUVECs were infected with either control AdGFP or AdIL-32β, for 36 hr followed by incubation with IL-1β at the dose indicated for 4 hr. THP-1 leukocyte adhesion was measured 30 minutes after incubation (FIG. 16B). The experiments were repeated at least 3 times. The data were presented as mean±SEM. *p<0.05.

FIG. 17: Diagram of IL-32β in vascular inflammation.

FIG. 18: IL-32β impairs wound healing. Two full-thickness excisional wounds were made in the dorsal paravertebral region of 8 weeks old mice with a 6 mm-diameter punch (Acu-Punch). The diameter of the wound was measured using caliper on post-wounding days 3, 6, and 9. Representative images were shown on post-wounding day 6. Arrows point the skin wound. Percentage of wound healing was calculated 3 days after creation of the wound. n=18 mice per group, *p<0.05.

FIG. 19: Non-invasive functional imaging of inflammation during wound healing in live animals. Excisional wounds were created in control HLL mice and IL-32-HLL mice. Three days later, the mice received IP injection of luciferin, followed by imaging of luciferase activity using an IVIS-200 machine. There are significantly higher levels of photon counts in IL-32-HLL mouse wounds than the ones from control HLL mice. Red circles indicate wound area. n=2 mice per group.

FIG. 20: IL-32β impairs skin hair growth. Hair was removed with clippers and Nair™ in wild-type and IL-32β transgenic mice, following by creating skin wounds. Hair growth and wound healing images were taken three days after the procedure (left panel). Skin tissues were harvested, fixed, embedded in paraffin and sectioned. Tissue sections were processed for immunohistochemical staining with an antibody against CD68, followed by incubation with 2^{nd} antibody conjugated with HRP. CD68-positive cells (macrophage) were detected with peroxidase activity with diaminobenzidine (brown color). The tissues were counted with hematoxylin (right panel).

### DETAILED DESCRIPTION OF THE PREFERRED EMBODIMENTS

### I. The Present Invention

The inventor herein reports a role for IL-32 in vascular inflammation and sepsis development, as well as in cancer and hair growth. It was found that IL-32β, in particular, is predominantly expressed in human endothelial cells, and inflammatory cytokines regulate its expression. IL-32β amplifies and sensitizes inflammatory cytokine-induced vascular inflammation through induction of inflammatory cytokines and cell adhesion molecules and subsequently leukocyte adhesion. In in vivo analysis using transgenic mice, the inventor confirmed critical functions of IL-32 in inflammation and sepsis development, and also identified elevated IL-32β in certain cancers. It was also observed that IL-32β correlated with loss inhibition or hair growth. Thus, these studies identifies a potentially important mediator of vascular inflammation and other human disease.

The cloning of natural killer cell transcript 4 (NK4; IL-32) permitted the inventor's analysis of expression in human endothelial cells by microarray analysis. Using Northern blots, Southern blots and RT-PCR, the inventor showed that IL-32 is expressed only in humans and certain closely related primates, but not in rodents and rabbits. Four splicing variants of IL-32 have been cloned. Among them, IL-32β is the major isoform in vascular endothelial cells. The inventor found that pro-inflammatory cytokines (TNFα and IL-1β) dramatically upregulate IL-32β expression in cultured endothelial cells in a NF-κB dependent manner, indicating a role for IL-32β in inflammation. Published results indicate IL-32β induces the degradation of IκBα, the inhibitor for NF-κB, in lymphocytes, illustrating a potential positive feedback regulation of IL-32β in vascular inflammation. Moreover, IL-32β sensitizes endothelial response to IL-1β and TNFα treatment in cell adhesion molecule expression as well as inflammatory cytokine production. As is known, during disease initiation, leukocytes must first adhere to inflamed microvasculature and then transmigrate through the vessel wall before cells can accumulate in the target tissues. Thus, the interaction of inflammatory cells with inflamed endothelium is a paramount process in disease development. Based on these findings, the inventor hypothesizes that IL-32β plays a role in vascular inflammation and inflammation related disease progression. To further characterize the role of IL-32β in vascular inflammation in vivo, the inventor has generated a transgenic mouse model (PECAM-IL32), in which IL-32β is driven by an endothelial specific, platelet endothelial cell adhesion molecule-1 (PECAM-1) promoter.

Involvement of inflammation in breast cancer has been shown, as well as in many other cancers. Thus, the inventor analyzed IL-32β expression in human breast cancer samples by real time quantitative RT-PCR. He found an increase of IL32β expression in tumor samples compared to normal controls, as well as a correlation of IL-32β levels with brain tumor malignancy. Using a transgenic animal, the inventor found an acceleration of tumor growth and progression, with tumor onset occurring at an much early age in the IL-32β transgenic mice compared to wild-type controls.

Chronic inflammation also is a risk factor for wound healing and hair growth. Wound healing is a process that involves both inflammation and the resolution of the inflammatory response, which culminates in remodeling. Uncontrolled or prolonged inflammation often leads to impaired wound healing that is a common complication of diabetes. Based on the findings of IL-32β in vascular inflammation and inflammation is a risk factor in diabetes, the inventor sought to determine whether IL-32β plays a role in diabetic wound healing and hair growth by regulating inflammation. The inventor observed a significant delay in wound healing in IL-32 mice, and a dramatic impairment of hair growth in IL-32β transgenic mice. Collectively, these findings suggest IL-32β enhances inflammation that can lead to impaired would healing and hair growth.

### II. IL-32β

As discussed above, roles for IL-32 in inflammation, cancer and hair growth have been identified. The invention contemplates the use of the entire IL-32β molecule, but also relates to fragments of the polypeptide that may or may not retain aspects of IL-32β activity. Fragments, including the N-terminus of the molecule may be generated by genetic engineering of translation stop sites within the coding region (discussed below). Alternatively, treatment of the protein with proteolytic enzymes, known as proteases, can produces a variety of N-terminal, C-terminal and internal fragments. Examples of fragments may include contiguous residues of the IL-32β sequence given in SEQ ID NO:2 of 6, 7, 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, 125, 150, or 178 amino acids in length. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

A. Structural Features of the Polypeptide

IL-32 was first cloned as natural killer cell transcript 4 (NK4) in 1992 and shown to be expressed in IL-2 activated natural killer (NK) cells and T cells (Dahl et al., 1992). Recently, the gene was renamed interleukin-32 (IL-32), due to its ability to induce various cytokines production such as TNFα and IL-8, although it does not share sequence homology with any known cytokine family proteins (Kim et al., 2005). The IL-32 gene has a full length of 705 bp and a predicted a molecular weight of 27 kDa. IL-32 exists as four splice variants in blood cells, named IL-32α, -β, -γ (full length) and -δ, with IL-32α (IL-32) being the major isoform in blood cells (Kim et al., 2005). The IL-32 peptide contains a RGD motif, indicating that it may be involved in cell-cell adhesion. The highest homology to human IL-32 was found in bovine tissue at 31.8%, and no homologue to this gene was found in mice and rats.

B. Functional Aspects

Since IL-32 expression is regulated by inflammatory cytokines in human peripheral lymphocytes, it has been speculated that it may play a role in inflammatory/autoimmune diseases (Kim et al., 2005). Further analysis shows an increase of IL-32 expression in inflammatory diseases, such as rheumatoid arthritis (Cagnard et al., 2005; Joosten et al., 2006; Shoda et al., 2006) and Crohn's disease (Netea et al., 2005). It was reported that cleavage of IL-32 by proteinase 3 (PR3) enhanced IL-8 secretion (Uehara et al., 2004). Proinflammatory cytokines such as IL-1β and TNFα also increase PR3 expression (Uehara et al., 2004), and furthermore, PR3 is internalized in endothelial cells and induces apoptosis (Yang et al., 2001). These results suggest inhibition of PR3 activity may modulate inflammation by inhibiting IL-32 activity as well as by inhibiting IL-1β or TNFα activity. In addition to its link with IL-1β and TNFα, a link between IL-32 and bacterial or viral infection was observed. Stimulation of blood cells from healthy donors with Mycobacterium tuberculosis or Mycobacterium bovis BCG increased IL-32 expression (Netea et al., 2005). IL-32 was also highly expressed in B-lymphocytes stimulated with Epstein-Barr virus (Carter et al., 2002).

In clinical studies, the levels of IL-32 in synovial tissues from rheumatoid arthritis (RA) patients correlates with macroscopic scoring, inflammation and cytokine levels. Microarray analysis of fibroblast-like synoviocytes obtained from patients with rheumatoid arthritis and those with non-inflammatory osteoarthritis showed a dramatic elevation of IL-32 in rheumatoid arthritis samples (Cagnard et al., 2005). Collectively, these findings suggest a role of IL-32 in inflammation and disease progression. Reducing IL-32 activity may benefit patients with rheumatoid arthritis and other inflammatory-related diseases.

Although, IL-32α/β/γ was reported to be closely associated with TNFα and exacerbates TNFα-induced inflammatory diseases, all the published studies are focused on blood cells such as monocytes and lymphocytes. The invenotor's data show that IL-32 is highly expressed in vascular endothelial cells, and its expression is strongly regulated by pro-inflammatory cytokines. In addition, there is now molecular evidence suggesting a role of IL-32β in vascular inflammation, an important event in disease progression. Other data now show that IL-32β increases the production of proinflammatory cytokines and adhesion molecules in endothelial cells, indicating a role of IL-32β in vascular inflammation. IL-32β may be a potential therapeutic target to control inflammation in human diseases. In addition, the inventors postulates that overexpression of IL-32β in vascular endothelium will render the mice sensitive to vascular inflammation and sepsis development.

C. Variants of IL-32β

The present invention contemplates that variation in the sequence of IL-32β can be tolerated without loss of function. Alternatively, other mutants of IL-32β that have lost some or even all activity may nonetheless be useful, for example, as targeting agents and as antigens. The following provides a general discussion of variants that may be applied, in various ways, to IL-32β.

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent or improved molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below. Table 1 shows the codons that encode particular amino acids.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making substitutional variants, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See, for example, Johnson et al., (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of Killin, but with altered and even improved characteristics.

C. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

D. Purification of Proteins

It will be desirable to purify IL-32β or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fuctose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Synthetic Peptides

The present invention also describes smaller IL-32β-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

F. Antibodies

In certain aspects of the invention, antibodies may find use as inhibitors of IL-32β activity. As used herein, the term “antibody” is intended to refer broadly to any appropriate binding agent such as IgG, IgM, IgA, IgD and IgE. Generally, IgG and/or IgM are preferred because they are the most common antibodies in the physiological situation and because they are most easily made in a laboratory setting.

The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

Single-chain antibodies are described in U.S. Pat. Nos. 4,946,778 and 5,888,773, each of which are hereby incorporated by reference.

“Humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

For detection of IL-32β protein sequences, a diagnostic kit of the present invention comprises, in one or more containers, an anti-IL-32β antibody which optionally can be detectably labeled. In a different embodiment, the kit can comprise in a container, a labeled specific binding portion of an antibody. As used herein, the term detectable label refers to any label which provides directly or indirectly a detectable signal and includes, for example, enzymes, radiolabelled molecules, fluorescent molecules, particles, chemiluminesors, enzyme substrates or cofactors, enzyme inhibitors, or magnetic particles. Examples of enzymes useful as detectable labels in the present invention include alkaline phosphatase and horse radish peroxidase. A variety of methods are available for linking the detectable labels to proteins of interest and include for example the use of a bifunctional agent, such as, 4,4′-difluoro-3,3′-dinitro-phenylsulfone, for attaching an enzyme, for example, horse radish peroxidase, to a protein of interest. The attached enzyme is then allowed to react with a substrate yielding a reaction product which is detectable.

The present invention provides a method for detecting an IL-32β protein in a patient sample, comprising, contacting the patient sample with an anti-IL-32β antibody under conditions such that immunospecific binding can occur, and detecting or measuring the amount of any immunospecific binding by the antibody. The method can be performed in situ in PMBCs, in cell lysate such as an ELISA, or cell lysate or purified protein in a western blot.

### III. Nucleic Acids

The present invention also provides, in another embodiment, genes encoding IL-32β. A gene for the human IL-32β molecule has been identified, and is included herein as SEQ ID NO:1. The present invention is not limited in scope to this gene, however, as homologs have been found in closely related primates such as gorilla and chimp.

In addition, it should be clear that the present invention is not limited to the specific nucleic acids disclosed herein. As discussed below, a “IL-32β gene” may contain a variety of different bases and yet still produce a corresponding polypeptide that is functionally indistinguishable from, and in some cases structurally identical to, the human gene disclosed herein.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the function of IL-32β.

A. Nucleic Acids Encoding IL-32β

Nucleic acids according to the present invention may encode an entire IL-32β gene, a domain of IL-32β that expresses a tumor suppressing function, or any other fragment of the IL-32β sequences set forth herein. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given IL-32β from a given species may be represented by natural variants that have slightly different nucleic acid sequences but, nonetheless, encode the same protein (see Table 1, above).

As used in this application, the term “a nucleic acid encoding a IL-32β” refers to a nucleic acid molecule that has been isolated free of total cellular nucleic acid. In certain embodiments, the invention concerns a nucleic acid sequence essentially as set forth in SEQ ID NO:1. The term “as set forth in SEQ ID NO:1” means that the nucleic acid sequence substantially corresponds to a portion of SEQ ID NO:1. The term “functionally equivalent codon” is used herein to refer to codons that encode the same amino acid, such as the six codons for arginine or serine, and also refers to codons that encode biologically equivalent amino acids, as discussed in the following pages.

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NO:1. Sequences that are essentially the same as those set forth in SEQ ID NO:1 also may be functionally defined as sequences that are capable of hybridizing to a nucleic acid segment containing the complement of SEQ ID NO:1 under standard conditions. It also is contemplated that the nucleic acid sequence is defined as that encoding SEQ ID NO:2, which permits variation in the use of codons to achieve the same amino acids sequence.

The DNA segments of the present invention include those encoding biologically functional equivalent IL-32β proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

B. Oligonucleotide Probes and Primers

Naturally, the present invention also encompasses DNA segments that are complementary, or essentially complementary, to the sequence set forth in SEQ ID NO:1. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 under relatively stringent conditions such as those described herein. Such sequences may encode the entire IL-32β protein or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to IL-32β or, more particularly, homologs of IL-32β from other species. Normally, the target DNA will be a genomic or cDNA library, although screening may involve analysis of RNA molecules. By varying the stringency of hybridization, and the region of the probe, different degrees of homology may be discovered.

C. Antisense Constructs

In some cases, it may be desirable to “knock-down” expression of IL-32β. Antisense treatments are one way of addressing this situation. Antisense technology also may be used to reduce expression, and hence function, of IL-32β in the development of cell lines or transgenic mice for research, diagnostic and screening purposes.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA'S, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

D. Ribozymes

Another “knock-down” approach involves the use of ribozymes, RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

E. Interfering RNAs

RNA interference (also referred to as “RNA-mediated interference” or RNAi) is another mechanism by which IL-32β expression could be modulated in a way similar to that of the antisense methodology. One can envision instances when inhibitory RNAs could be reduced or eliminated, leading to increased expression of IL-32β. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000). siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides +3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25 mer lengths with the endogenous nuclease complex that converts long dsRNAs to siRNAs in vivo. They do not describe or present data for synthesizing and using in vitro transcribed 21-25 mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

### IV. Diagnosing Cancers

IL-32β and the corresponding gene may be employed as a diagnostic or prognostic indicator of cancer. More specifically, overexpression of IL-32β is at least indicative of cancer or cancer risk, and may itself be a causative factor.

A. Genetic Diagnosis

One embodiment of the instant invention comprises a method for detecting variation in the expression of IL-32β. This may comprises determining that level of IL-32β. Obviously, this sort of assay has importance in the diagnosis of related cancers. Such cancer may involve cancers of the brain (glioblastomas, medulloblastoma, astrocytoma, oligodendroglioma, ependymomas), lung, liver, spleen, kidney, pancreas, small intestine, blood cells, lymph node, colon, breast, endometrium, stomach, prostate, testicle, ovary, skin, head and neck, esophagus, bone marrow, blood or other tissue.

The biological sample can be any tissue or fluid. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow or kidney. Other embodiments include fluid samples such as peripheral blood, lymph fluid, ascites, serous fluid, pleural effusion, sputum, cerebrospinal fluid, lacrimal fluid, stool or urine.

Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have IL-32β-related pathologies. In this way, it is possible to correlate the amount or kind of IL-32β detected with various clinical states.

(i) Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process. In particular embodiments, the probes or primers are labeled with radioactive species (³²P, ¹⁴C, ³⁵S, ³H, or other label), with a fluorophore (rhodamine, fluorescein) or a chemillumiscent (luciferase).

(ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention, Walker et al. (1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double-stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single-stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide,” thereby amplifying the di-oligonucleotide, may also be used in the amplification step of the present invention. Wu et al., (1989), incorporated herein by reference in its entirety.

(iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

(iv) Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

(v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols (Sambrook et al. 1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the IL-32β gene that may then be analyzed by direct sequencing.

(vi) Design and Theoretical Considerations for Relative Quantitative RT-PCR™

Reverse transcription (RT) of RNA to cDNA followed by relative quantitative PCR™ (RT-PCR™) can be used to determine the relative concentrations of specific mRNA species isolated from patients. By determining that the concentration of a specific mRNA species varies, it is shown that the gene encoding the specific mRNA species is differentially expressed.

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ experiment to successfully determine the relative abundances of a particular mRNA species is that relative concentrations of the amplifiable cDNAs must be normalized to some independent standard. The goal of an RT-PCR™ experiment is to determine the abundance of a particular mRNA species relative to the average abundance of all mRNA species in the sample. In the experiments described below, mRNAs for β-actin, asparagine synthetase and lipocortin II were used as external and internal standards to which the relative abundance of other mRNAs are compared.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR™ assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR™ is performed as a relative quantitative RT-PCR™ with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR™ assay with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR™ assays can be superior to those derived from the relative quantitative RT-PCR™ assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR™ product, display of the product on an electrophoretic gel or another display method becomes less complex, has less background and is easier to interpret.

(vii) Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al. (1994); Fodor et al. (1991).

B. Immunodiagnosis

Antibodies of the present invention can be used in characterizing the IL-32β content of healthy and diseased tissues, through techniques such as ELISAs and Western blotting. This may provide a screen for the presence or absence of malignancy or as a predictor of future cancer.

The use of antibodies of the present invention, in an ELISA assay is contemplated. For example, anti-IL-32β antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for IL-32β that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

C. Diagnostic Kits

All the essential materials and reagents required for detecting IL-32β nucleic acids may be assembled together in a kit. This generally will comprise preselected primers and probes, and may also include enzymes suitable for amplifying nucleic acids including various polymerases (RT, Taq, Sequenase™, etc.), deoxynucleotides and the like. Such kits also generally will comprise, in suitable means, distinct containers for each individual reagent and enzyme as well as for each primer or probe.

Also provided are kits for detecting IL-32β protein expression. The kit of the present invention can be prepared by known materials, such as an IL-32β monoclonal antibody or polyclonal serum. Again, kits comprises separate vials or containers for the various reagents, such as antibodies and detection reagents, etc.

The reagents are also generally prepared in a form suitable for preservation by dissolving it in a suitable solvent. Examples of a suitable solvent include water, ethanol, various buffer solutions, and the like. The various vials or containers are often held in blow-molded or injection-molded plastics.

### V. Methods of Therapy

The present invention also involves, in another embodiment, the treatment of cancer. The types of cancer that may be treated, according to the present invention, is limited only by the involvement of IL-32β. Thus, it is contemplated that a wide variety of tumors may be treated using anti-IL-32β therapy, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

A. Nucleic Acid-Based Therapies

One of the therapeutic embodiments contemplated by the present inventors is the intervention, at the molecular level, in the events involved in tumorigenesis. Specifically, the present inventors intend to provide, to a cancer cell, a nucleic acid (ribozyme, antisense, siRNA) capable of inhibiting IL-32β activity in that cell. Because the sequence homology between the human and other primates, any of these nucleic acids could be used in human therapy, as could any of the gene sequence variants discussed above which would encode the same, or a biologically equivalent polypeptide. The lengthy discussion of expression vectors and the genetic elements employed therein is incorporated into this section by reference. Particularly preferred expression vectors are viral vectors such as adenovirus, adeno-associated virus, herpesvirus, vaccinia virus and retrovirus. Also preferred is liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various tumor types. The section below on routes contains an extensive list of possible routes. For practically any tumor, systemic delivery is contemplated. This will prove especially important for attacking microscopic or metastatic cancer. Where discrete tumor mass may be identified, a variety of direct, local and regional approaches may be taken. For example, the tumor may be directly injected with the expression vector. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

In a different embodiment, ex vivo gene therapy is contemplated. This approach is particularly suited, although not limited, to treatment of bone marrow associated cancers. In an ex vivo embodiment, cells from the patient are removed and maintained outside the body for at least some period of time. During this period, a therapy is delivered, after which the cells are reintroduced into the patient; hopefully, any tumor cells in the sample have been killed.

Autologous bone marrow transplant (ABMT) is an example of ex vivo gene therapy. Basically, the notion behind ABMT is that the patient will serve as his or her own bone marrow donor. Thus, a normally lethal dose of irradiation or chemotherapeutic may be delivered to the patient to kill tumor cells, and the bone marrow repopulated with the patients own cells that have been maintained (and perhaps expanded) ex vivo. Because, bone marrow often is contaminated with tumor cells, it is desirable to purge the bone marrow of these cells. Use of gene therapy to accomplish this goal is yet another way IL-32β may be utilized according to the present invention.

B. Antibody Therapy

Another therapy approach is the provision, to a subject, of a polypeptide that binds to and inactivates IL-32β. The antibody may be produced by recombinant expression means. Formulations would be selected based on the route of administration and purpose including, but not limited to, liposomal formulations and classic pharmaceutical preparations.

C. Combined Therapy with Immunotherapy, Traditional Chemo- or Radiotherapy

Tumor cell resistance to DNA damaging agents represents a major problem in clinical oncology. One goal of current cancer research is to find ways to improve the efficacy of chemo- and radiotherapy. One way is by combining such traditional therapies with gene therapy. For example, the herpes simplex-thymidine kinase (HS-tk) gene, when delivered to brain tumors by a retroviral vector system, successfully induced susceptibility to the antiviral agent ganciclovir (Culver et al., 1992). In the context of the present invention, it is contemplated that an anti-IL-32β therapy could be used similarly in conjunction with chemo- or radiotherapeutic intervention. It also may prove effective to combine anti-IL-32β therapy with immunotherapy.

To kill cells, inhibit cell growth, inhibit metastasis, inhibit angiogenesis or otherwise reverse or reduce the malignant phenotype of tumor cells, using the methods and compositions of the present invention, one would generally contact a “target” cell with an anti-IL-32β therapeutic and at least one other agent. These compositions would be provided in a combined amount effective to kill or inhibit proliferation of the cell.

This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

Alternatively, the gene therapy treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more particularly within about 6-12 hours of each other, with a delay time of only about 12 hours being particularly contemplated. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either Killin or the other agent will be desired. Various combinations may be employed, where the anti-IL-32β is “A” and the other agent is “B,” as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated. Again, to achieve cell killing, both agents are delivered to a cell in a combined amount effective to kill the cell.

Agents or factors suitable for use in a combined therapy are any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide. In certain embodiments, the use of cisplatin in combination with an anti-IL-32β therapy is particularly contemplated.

In treating cancer according to the invention, one would contact the tumor cells with an agent in addition to the expression construct. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or more preferably, cisplatin. The agent may be prepared and used as a combined therapeutic composition, or kit, by combining it with an anti-IL-32β therapy, as described above.

Agents that directly cross-link nucleic acids, specifically DNA, are envisaged to facilitate DNA damage leading to a synergistic, antineoplastic combination with an anti-IL-32β therapy. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the local or regional delivery of an anti-IL-32β therapy to patients with cancer will be a very efficient method for treating the clinical disease. Similarly, the chemo- or radiotherapy may be directed to a particular, affected region of the subjects body. Alternatively, systemic delivery of expression construct and/or the agent may be appropriate in certain circumstances, for example, where extensive metastasis has occurred.

In addition to combining IL-32β-directed therapies with chemo- and radiotherapies, it also is contemplated that combination with other gene therapies will be advantageous. For example, gene replacement therapy for p21, Rb, APC, DCC, NF-1, NF-2, BCRA2, p16, FHIT, WT-1, MEN-I, MEN-II, BRCA1, VHL, FCC, MCC, ras, myc, neu, raf, erb, src, fms, jun, trk, ret, gsp, hst, bcl and abl.

It also should be pointed out that any of the foregoing therapies may prove useful by themselves in treating a cancer. In this regard, reference to chemotherapeutics and non-IL-32β therapy in combination should also be read as a contemplation that these approaches may be employed separately.

E. Formulations and Routes for Administration to Patients

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or administration local or regional to a tumor, for example, in the local or regional vasculature or lymphatic system, or in a resected tumor bed.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VI. Restoration of Hair Growth and Promotion of Wound Healing

The present invention also provides for the restoration/promotion of hair growth and promotion of wound healing. The methods and IL-32β antagonists described above for treatment of cancer will be applied in these settings as well, except that additional embodiments for delivery to wound sites and hair surfaces (e.g., scalp) will be of particular interest. Thus, topical applications involving creams, gels, etc. will be utilized, as well the incorporation of IL-32β antagonists into dressings/wound coverings, such as bandages, gauze, mesh, and other wound coatings. Such delivery modes may be combined in time or physically with second agents, such as other hair growth promoters (Minoxidil®) or wound healing agents (antiseptics, antibiotics, anti-scarring agents, anti-inflammatory agents, moisturizing agents). Repeated, continuous or time-delayed delivering methods also will be used.

### VII. Transgenics

In one embodiment of the invention, transgenic animals are produced which express excess amounts of IL-32β or, alternatively, are “knocked-out” for expression of IL-32β. Transgenic animals having such phenotypes, and recombinant cell lines derived from such animals, will find use in various models of disease, and thus for screening against candidate therapies. Trans genic animals of the present invention also can be used as models for studying indications such as cancers or hair loss. The promoter controlling the transgene may be one that is capable of tissue-specific or -inducible expression, in particular, in vascular endothelium.

The transgenic animal is produced by the integration of the transgene into the genome in a manner that permits the expression of the trans gene. Methods for producing transgenic animals are generally described by Wagner and Hoppe (U.S. Pat. No. 4,873,191; which is incorporated herein by reference), Brinster et al. (1985; which is incorporated herein by reference in its entirety) and in “Manipulating the Mouse Embryo; A Laboratory Manual” (1994); which is incorporated herein by reference in its entirety).

It may be desirable to replace the endogenous IL-32β by homologous recombination between the transgene and the endogenous gene so as to measure the effects of only the transgene's expression. Typically, a IL-32β gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Alternatively, the endogenous gene may be eliminated by deletion as in the preparation of “knock-out” animals, optionally followed by insertion of the IL-32β transgene. The absence of one or both alleles of a IL-32β gene in “knock-out” mice permits the study of the effects that a reduction in or loss of IL-32β protein has on a cell in vivo.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Sepsis/Materials & Methods

Cell culture. Human umbilical vein endothelial cells (HUVECs), human aortic smooth muscle cells (HASMCs), and human cardio aortic endothelial cells (HCAECs) were purchased (Clonetics, San Diego, Calif.), and grown on 0.1% gelatin-coated plates in endothelial growth medium, EGM (Clonetics). MCF-7, HT29, U251MG, H460, DU145, HEK293, CCD21-SK cells were grown in DMEM with 10% FBS, and HL60, THP-1 cells were grown in RPM11640 with 10% FBS. The adenoviral vectors directing the expression of IκB (AdIκBα), a mutated IκB as a NF-κB inhibitor, Akt (AdAkt) and a dominant-negative Akt1 (Addn-Akt) (Kotani et al., 1999), as well as LacZ (Adβ-gal) and GFP (AdGFP) as viral vector controls were used (Jobin et al., 1998). Viral vectors were propagated in 293 cells and purified by CsCl column (Lin et al., 1998).

Mice. The inventor constructed a plasmid in which IL-32β is driven by an endothelial specific, platelet endothelial cell adhesion molecule-1 (PECAM-1) promoter (Terry et al., 1997). Using the plasmid a transgenic mouse line was generated in FVB background at the Vanderbilt Transgenic Mouse/Embryonic Stem Cell Shared Resource. The expression of the gene was confirmed using qRT-PCR in the ECs isolated from lungs as described before (Kamiyama et al., 2006) and bone marrow cells.

IL-32 cDNA Synthesis and Cloning. The cDNAs were synthesized from cultured HUVECs. The cDNAs were used to amplify IL-32 using the specific primer set: 5′-GGGAATTCATGTGCTTCCCGAAGGTC-3′ (forward) (SEQ ID NO:3) and 5′-GGCTCGAGTCATTTTGAGGATTGGGG-3′ (reverse) (SEQ ID NO:4). PCR products were cloned into the EcoRI and XhoI sites of pcDNA3.1/V5-HisC (Invitrogen, Carlsbad, Calif.), and sequenced. IL-32β was also cloned into pAdTrack-CMV and packaged with pAdEasy-1 for adenovirus preparation (Ad-IL-32β) and into the pEGFP-N3 vector for intracellular imaging (BD Biosciences, Mountain View, Calif.).

Tissue Distribution Analysis. Tissue distribution of IL-32 was examined using multiple tissue cDNA panels (Clontech, Mountain View, Calif.). IL-32 was amplified using the following specific primer sets: 5′-ATGTGCTTCCCGAAGGTCCTCTCTGA-3′ (forward) (SEQ ID NO:5), and 5′-TCATTTTGAGGATTGGGGTTCAGAGC-3′ (reverse) (SEQ ID NO:6). Glyceraldehyde 3-phosphate dehydrogenase (G3PDH) was used as an internal control. The amplified products were electrophoresed on 2% (w/v) agarose gel.

Northern analysis. Cells were infected with adenoviral vectors expressing genes of interest for 48 hour. Cells were then serum starved for 5 hour, followed by treatment with IL-1β or TNF-α (R D Systems, Minneapolis, Minn.) at 10 ng/ml. RNA was isolated and subjected for Northern blot analysis. ³²P labeled cDNA probes for IL-32 mRNA were hybridized using Express Hyb (BD Biosciences).

Southern analysis. DNA was extracted from human, mouse and rat tissues using phenol/chloroform following the incubation of minced tissues in lysis buffer (50 mM Tris, pH8, 100 mM EDTA, 0.5% SDS, and 500 μg/ml proteinase K). A primate phylogenetic panel was used to obtain various primates' DNA (Coriell Institute for Medical Research, Camden, N.J.). Ten μg of DNA was digested with SacI overnight at 37° C. DNA was analyzed by the standard hybridization protocol with a probe that was prepared from the exon 7 of the gene.

ELISA and Western. Cell lysate and the medium were collected following the infection of HUVECs with AdGFP or AdIL-32β for 48 hrs. The medium was concentrated using Centriplus® YM-10 (Millipore Corporation, Bedford, Mass.). ELISA was performed using Ni-NTA HisSorb™ Strips (Qiagen, Valencia, Calif.) according to the manufacture's protocol. For Western blot, the lysate and the concentrated medium were immunoprecipitated with a V5 antibody and the samples were run in SDS-PAGE. Immunoblotting of the samples were performed with anti-V.5 antibody and a horseradish peroxidase conjugated anti-mouse secondary antibody (Promega, Madison, Wis.).

Akt Kinase Assays. Akt kinase assay was conducted using an Akt kinase assay kit (Cell Signaling Technology, Danvers, Mass.). qRT-PCR RNA was isolated from HUVECs infected with either AdGFP or AdIL-32β for 48 hr. Total RNA (1 μg) was used for the first-strand cDNA synthesis using iScript™ cDNA synthesis kit (Bio Rad, Hercules, Calif.). qPCR was done using IQ™ SYBR® Green supermix (Bio Rad) on iCycler (Bio Rad). 18S or β-actin was used for normalization of each gene. ICAM-1 primers were 5′-CCACAGTCACCTATGGCAAC-3′ (forward) (SEQ ID NO:7) and 5′-AGTGTCTCCTGGCTCTGGTT-3′ (reverse) (SEQ ID NO:8). VCAM-1 primers were 5′-GCTTCAGGAGCTGAATACCC-3′ (forward) (SEQ ID NO:9) and 5′-AAGGATCACGACCATCTTCC-3′ (reverse) (SEQ ID NO:10). E-selectin primers were 5′-TGAACCCAACAATAGGCAAA-3′ (forward) (SEQ ID NO:11) and 5′-CCTCTCATCATTCCACATGC-3′ (reverse) (SEQ ID NO:12). IL-1β primers were 5′-CCCAACTGGTACATCAGCAC (forward) (SEQ ID NO:13) and 5′-GGAAGACACAAATTGCATGG-3′ (reverse) (SEQ ID NO:14). TNFα primers were 5′-CCTCTTCTCCTTCCTGATCG-3′ (forward) (SEQ ID NO:15) and 5′-ATCACTCCAAAGTGCAGCAG-3′(reverse) (SEQ ID NO:16). IL-32 primers were 5′-CGACTTCAAAGAGGGCTACC-3′ (forward) (SEQ ID NO:17) and 5′-GAGTGAGCTCTGGGTGCTG-3′ (reverse) (SEQ ID NO:18).

Cell Imaging. Bovine aortic endothelial cells (BAVECs) were transfected with either the pEGFP-IL32β expression vector, or the empty vector using Lipofectamin 2000 (Invitrogen). The following day, the cells were incubated with ER-Tracker™ Red (Molecular Probes, Inc. Eugene, Oreg.) at 100 nM for 15 min prior to fixation with 4% formaldehyde. The cells were analyzed by LSM 510 Confocal microscopy at the VUMC Cell Imaging Shared Resource.

Cell Adhesion Assay. HUVECs that were infected with either AdGFP or AdIL-32β were treated with recombinant IL-1β for 4 hours. THP-1 cells that were labeled with Cell Tracker™ Red CMTPX (Molecular Probes, Eugene, Oreg.) were allowed to adhere to the monolayer of HUVECs for 30 min. Adhered THP-1 cells were counted under fluorescent microscope. Similarly, cell adhesion assay was performed using ECs isolated from PECAM-IL32 and WT mice.

Cecal-Ligation and Puncture (CLP) model. Ten week-old mice were used for CLP procedure. Briefly, mice abdomen were cleaned Mice were anesthetized with isoflurane gas. Through a midline incision, the cecum was exteriorized and ligated just below the ileocecal valve so that intestinal obstruction will not be produced. The cecum was then punctured twice using 20-gauge needle, and squeezed to ensure that the wounds will be open. The abdominal incision was sutured back in layers (Remick et al., 2005; Baker et al., 1983). Seventeen hours after CLP, mice were sacrificed and blood was drawn. Serum concentration of IL-1β and TNFα were measured using commercial ELISA kit (R&D system, Minneapolis, Minn.).

Statistics. Results are reported as mean±SEM. Statistical analysis was done using a two-tailed Student's t-test. Differences were considered statistically significant at p<0.05.

### Example 2

### Sepsis/Results

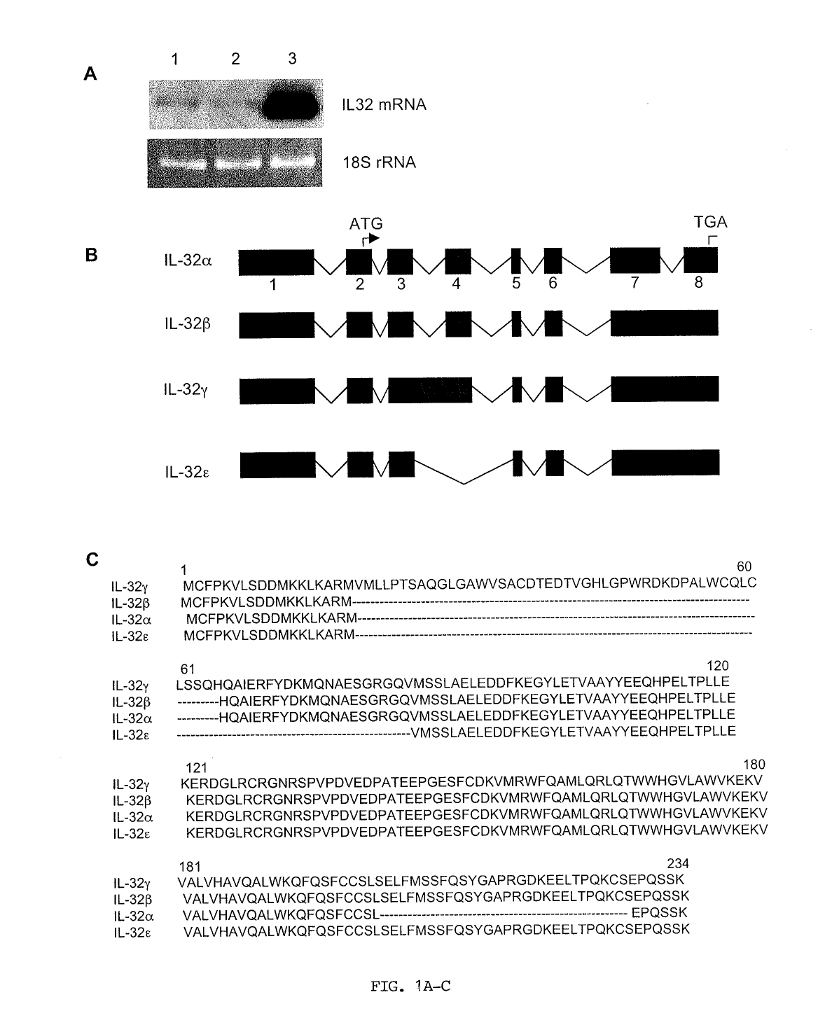

Cloning of IL-32 from human endothelial cells. In searching for Akt target genes in endothelial cells, the inventor performed a cDNA microarray analysis on Akt activated human endothelial cells (HUVECs). NK4, which was recently renamed as IL-32, was identified as one of the highly upregulated genes by activated Akt (data not shown). This result was further confirmed by Northern blot in cultured endothelial cells (FIG. 1A). The inventor cloned the gene and subsequent sequence analysis identified three isoforms of IL32, which are 489 bp (IL-32α); 567 bp (IL-32>); and 507 bp (IL-32γ) (FIGS. 1B-C). Among them, IL-32β is the major isoform in endothelial cells. This finding is in agreement with published data that multiple isoforms of IL-32 exist in hematopoietic derived cells, however, with IL-32β as the major isoform in those cells.

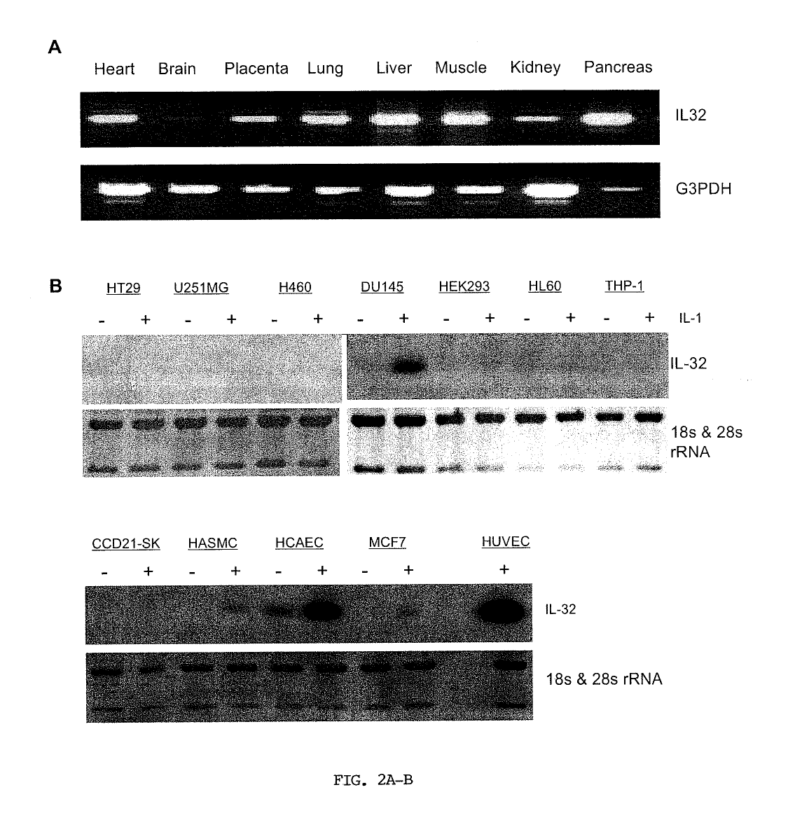

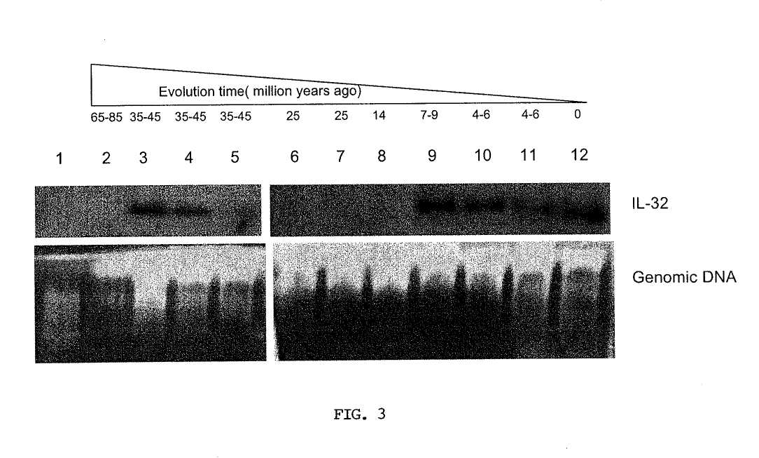

IL-32 is expressed in human and some primate tissues, and predominantly in endothelial cells. Genomic search for any homologues or known domains against different genome data banks indicates that IL-32 does not exist in any known genomic library of other species. Published data on hematopoietic cells confirmed this analysis and indicated the closed homology with bovine at 31% (Dahl et al., 1992; Kim et al., 2005; Goda et al., 2006). The inventor performed Northern blot and RT-PCR analysis and confirmed no existence of IL-32 in rodent tissues (data not shown). Therefore, the inventor focused on IL-32 expression in human tissues. Semi-quantitative PCR of IL-32 was performed using genomic cDNA from 8 different human organs. The results showed that IL-32 is ubiquitously expressed in most of the organs tested, with the highest levels in the lung, liver, skeletal muscle, pancreas and heart. Expression levels were low in the placenta and kidney, and hardly detectable in the brain (FIG. 2A).

Next, the inventor examined the cell type specific expression of IL-32. He performed Northern blot analysis for IL-32 expression on samples collected from a variety of cultured human cell lines, which include endothelial cells, epithelial cells, smooth muscle cells, fibroblast cells and leukocytes. These cells were treated with or without IL-1β. Interestingly, the inventor found that IL-32 is mainly expressed in human endothelial cells, including large and small vascular endothelial cells, and the inflammatory cytokine dramatically induced IL-32 expression in these cells. There is hardly any detectable amount of IL-32 in most non-endothelial cells, with a few exceptions (FIG. 2B). The prostate cancer cell line DU145 expresses IL-32 and responds to IL-1β stimulation. DU145 has great tendency to form vascular channels and exhibits vascular mimicry (data not shown). Currently, it is unclear whether such vascular mimicry has any bearing on IL-32 expression in this tumor line.

Since the genomic search in different data banks indicated that this gene does not exist in other species besides human, the inventor performed Northern blot, Southern blot and PCR analysis in rodent samples, the inventor were unable to detect any related sequences in rodents (data not shown). Furthermore, for analysis of IL-32 expression during evolution, the inventor collected primate DNA samples and analyzed for IL-32 expression by Southern blot. The results show that IL-32 is found in some primates that are close to us during evolution. It appears that IL-32 expression diverged into two branches during evolution with one branch at 7-9 million years ago and the other branch about 35-45 millions years ago (FIG. 3).

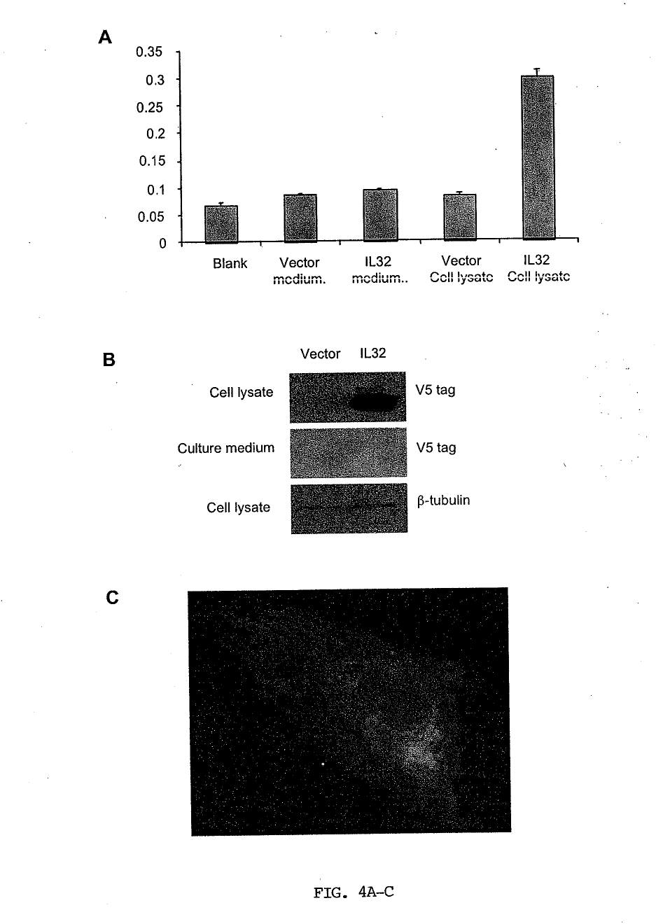

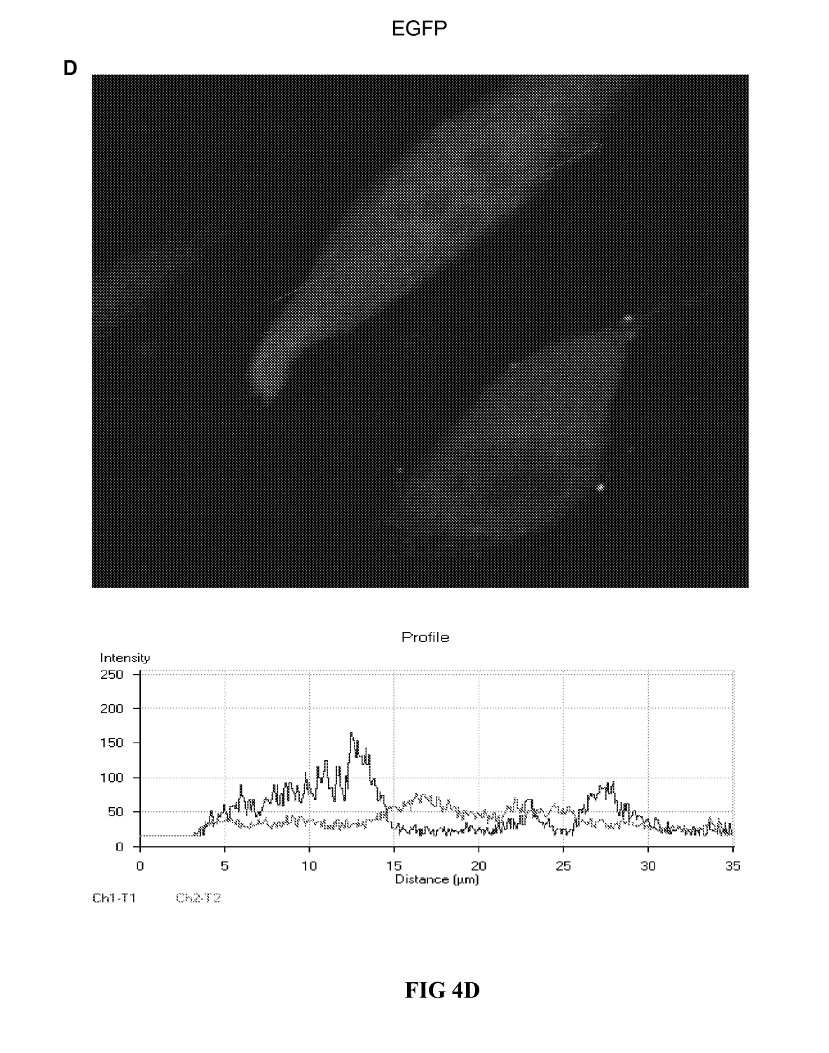

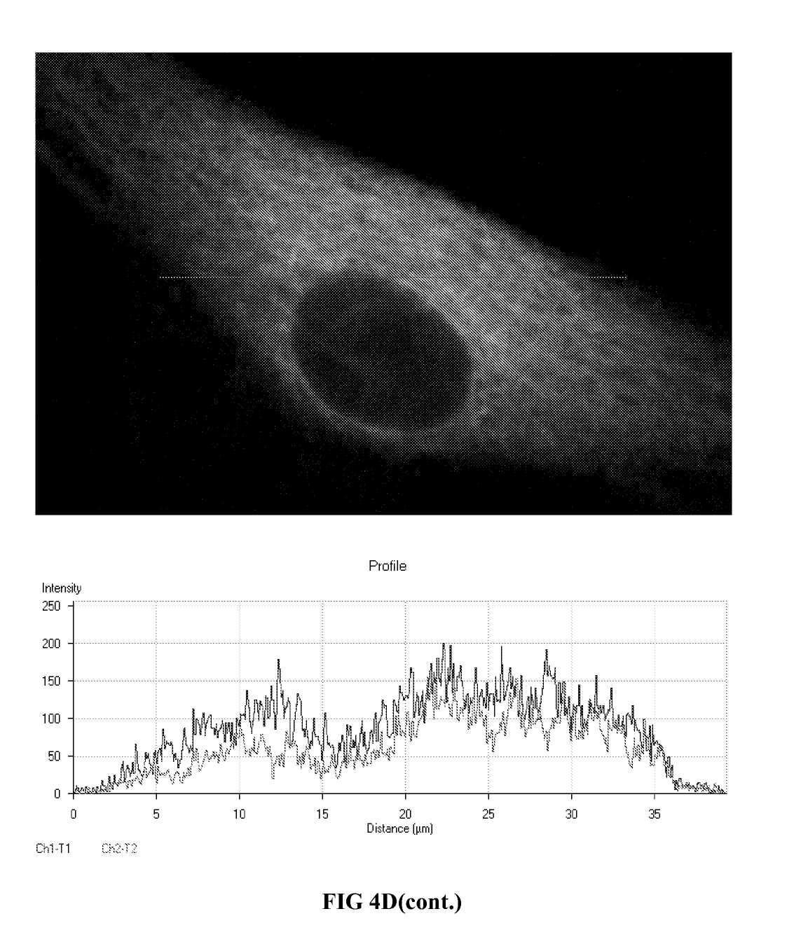

IL-32β is an intracellular protein and localizes in ER in endothelial cells. The inventor first investigated whether IL-32β, the major isoform found in endothelial cells, is a secreted protein. He generated an adenoviral vector directing the expression of IL-32β tagged with His and V5 epitopes (AdIL-32β) following the same protocol as previously described (Lin et al., 1998). The adenoviral vector allows for high efficiency of gene transfer and high levels of gene expression in endothelial cells (DeBusk et al., 2004); it is known that endothelial cells are very resistant to conventional cell transfection. HUVECs were infected with AdIL-32β or control vector for 48 hours. Cell lysate and concentrated cell culture supernatant were analyzed for the presence of IL-32β by ELISA and Western blot using specific antibodies against the His and V5 tags. Although the inventor clearly detected IL-32β expression in cell lysates infected with AdIL-32β, the inventor failed to detect any IL-32β in culture media by both methods (FIGS. 4A-B). This result suggests that IL-32β is an intracellular protein.

Next, the inventor analyzed the intracellular localization of IL-32β. He used conventional transfection over the adenoviral vector to avoid gene overexpression, and used bovine endotheial cell (BAVECs) over HUVECs for higher transfection efficiency. BAVECs were transfected with an expression vector (pEGFP-IL-32β), in which IL-32β was fused to the C terminus of EGFP. Empty vector (pEGFP-N3) was used as a control. 48 hours after cell transfection, IL-32β distribution visualized by EGFP expression shows that IL-32β is in the cytosol but not in the nucleus or at the plasma membrane (FIG. 4C). Further analysis using an ER specific tracking dye confirmed that IL-32β is localized in the ER (FIG. 4D).

IL-1β and TNFα induce IL-32 expression in a NF-κB-dependent manner. Since IL-32 is predominantly expressed in endothelial cells, the inventor reasoned that its potential functions might involve angiogenesis and vascular inflammation. First, the inventor tested whether angiogenic factors, such as VEGF, FGF, angiopoietin-1 and angiopoietin-2, could regulate IL-32 expression. The inventor found that none of these angiogenic factors affected IL-32 expression (data not shown). Furthermore, overexpression of IL-32β in endothelial cells did not affect cell proliferation, migration, or tubule formation, common assays for angiogenesis. Therefore, the inventors concluded that IL-32β unlikely plays a role in angiogenesis.

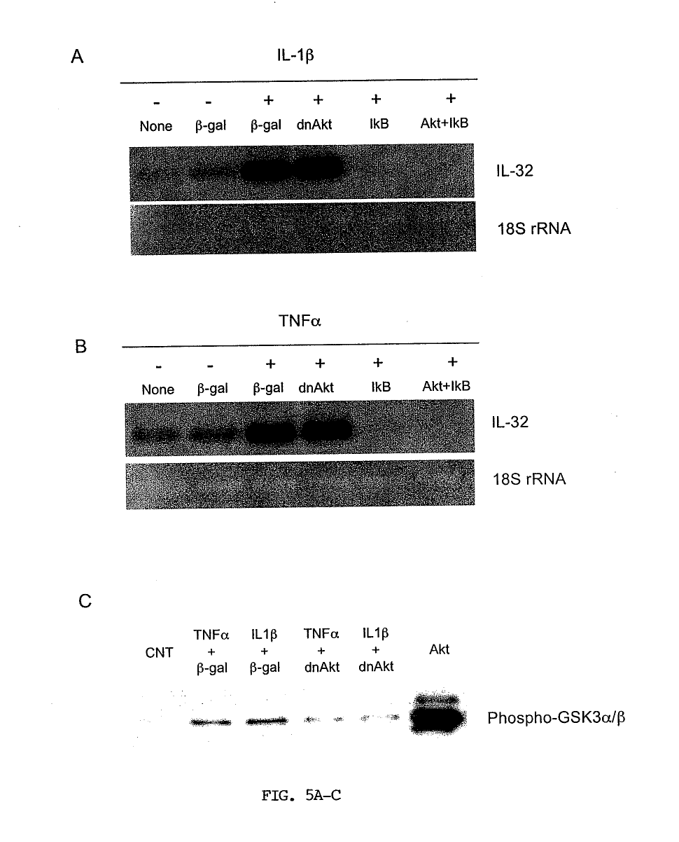

Next, the inventor examined its role in vascular inflammation. HUVECs were stimulated with recombinant IL-1β or TNFα, two major proinflammatory cytokines, for up to 12 hours. Cell lysates were analyzed for IL-32 expression. Both inflammatory cytokines dramatically induced IL-32 expression in endothelial cells (FIGS. 5A-B). Since the inventor originally discovered IL-32 as an Akt target gene in endothelial cells, and IL-1 and TNFα stimulation activates Akt, therefore the inventor determined the contribution of Akt activity in IL-32 induction. He found that although Akt is sufficient for IL-32 gene expression (FIG. 1A), Akt is not required for IL-32 induction in response to IL-1 and TNFα treatment. Addition of a dominant-negative Akt construct to block Akt activity (FIG. 5C) had no effect on inflammatory cytokine-induced IL-32 expression (FIGS. 5A-B).

NF-κB is a critical transcription factor that regulates gene expression involved in inflammation. The inventor therefore examined its role in IL-32 gene expression. He used a mutant IκB to block the activity of NF-κB activation (Jobin et al., 1998). These data show that blocking NF-κB activity completely abolished inflammatory cytokine-induced IL-32 expression in endothelial cells (FIGS. 5A-B). Taken together, these data demonstrate that inflammatory cytokines up-regulate IL-32 expression in a NF-κB transcription factor dependent manner.

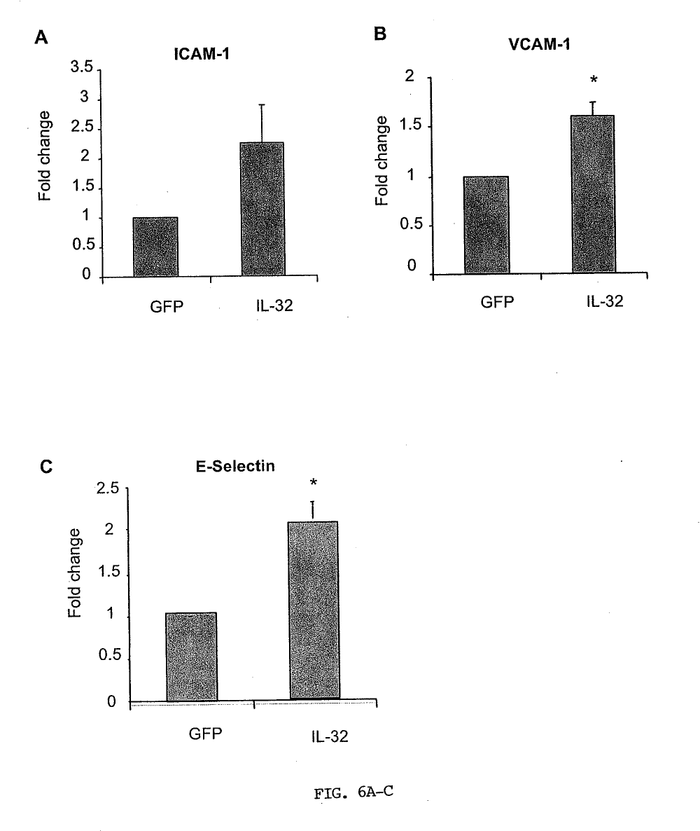

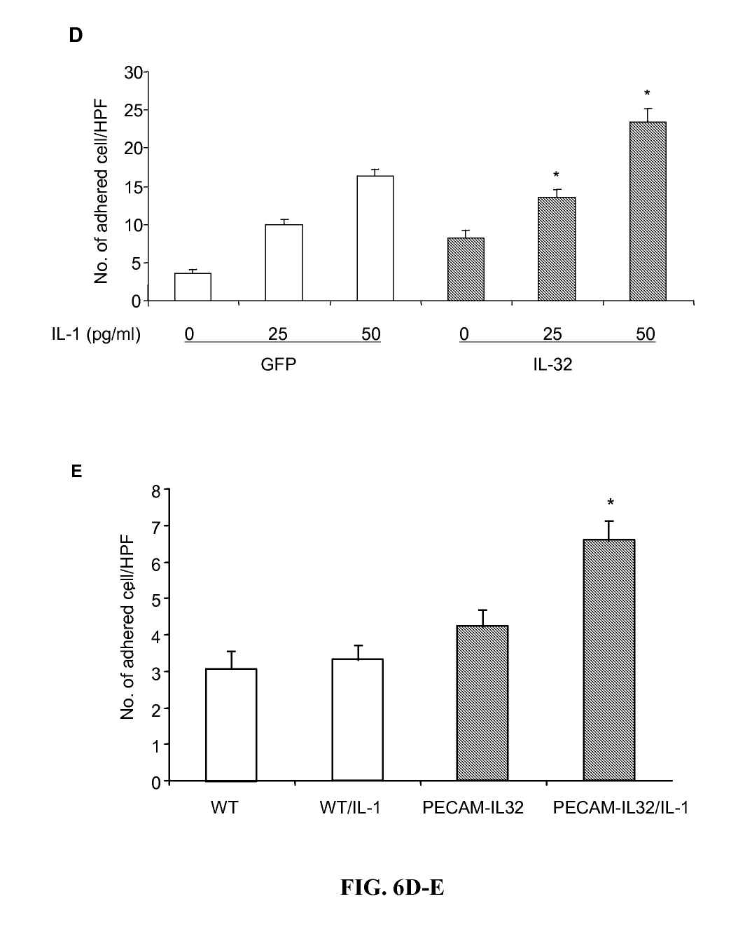

IL-32β enhances vascular cell adhesion molecule expression and vascular inflammation. Based on the findings that IL-32 is predominantly expressed in endothelial cells and proinflammatory cytokines regulate its expression, the inventor postulated that this gene might play a role in vascular inflammation. The expression of cell adhesion molecules, such as ICAM-1, VCAM-1, and E-selectin, on activated endothelium is an initial and critical event in vascular inflammation. The inventor found that IL-32β stimulation alone had no significant effects on the expression of these cell adhesion molecules on endothelial cells (data not shown). However, addition of IL-32β in endothelial cells with a very low dose of IL-1β stimulation enhanced the expression of ICAM-1, VCAM-1, and E-selectin compared to controls (FIGS. 6A-C), suggesting that IL-32β sensitizes and amplifies endothelial cells to inflammatory cytokine stimulation.

To further evaluate the biological consequences of the elevated levels of cell adhesion molecules in response to IL-32β, the inventor performed a leukocyte adhesion assay using monocytic cells, THP-1. In agreement with the high levels of cell adhesion molecules (FIGS. 6A-C), there were more THP-1 cells adhered to IL-32β transfected endothelial cells that were treated with a very low dose of IL-1β (FIG. 6D). Collectively, these data demonstrate a contribution of IL-32 to vascular inflammation.

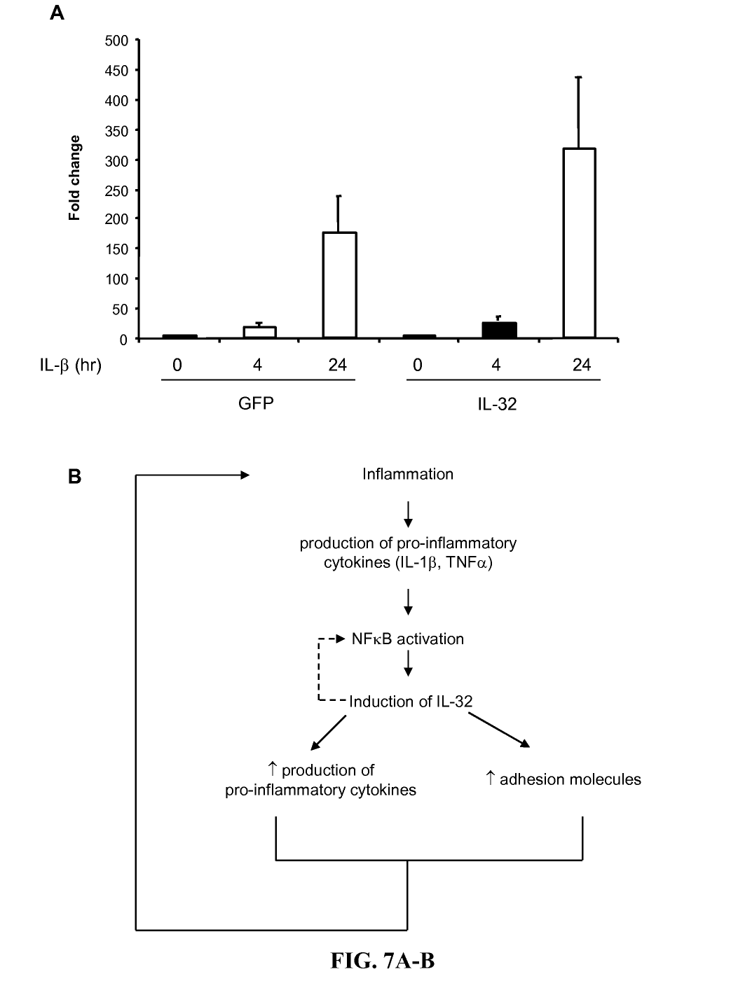

IL-32β upregulates the production of inflammatory cytokines. One of the interesting features of proinflammatory cytokines is that they induce their own expression and therefore amplify the inflammatory signal. Since the inventor found that IL-32β sensitizes endothelial cells to inflammatory cytokine stimulation (FIGS. 5A-D), he examined whether IL-32β also enhances cytokine expression. Endothelial cells were challenged with IL-1β in combination with IL-32β, followed by measurement of expression of IL-1β. The inventor observed an upregulation of IL-1β upon stimulation with IL-32β in combination with IL-1β compared to either treatment alone in endothelial cells (FIG. 7A). Identical results were observed in TNF-α treated cells (data not shown). Collectively, these results illustrate a positive feedback mechanism, in which inflammatory cytokines induce IL-32β expression via the NF-κB transcription factor and IL-32 is implicated causing degradation of IκB in hematopoietic cells (Kim et al., 2005), which further enhances the expression of cell adhesion molecules and proinflammatory cytokines (FIG. 7B). As a result, it amplifies and prolongs vascular inflammation.

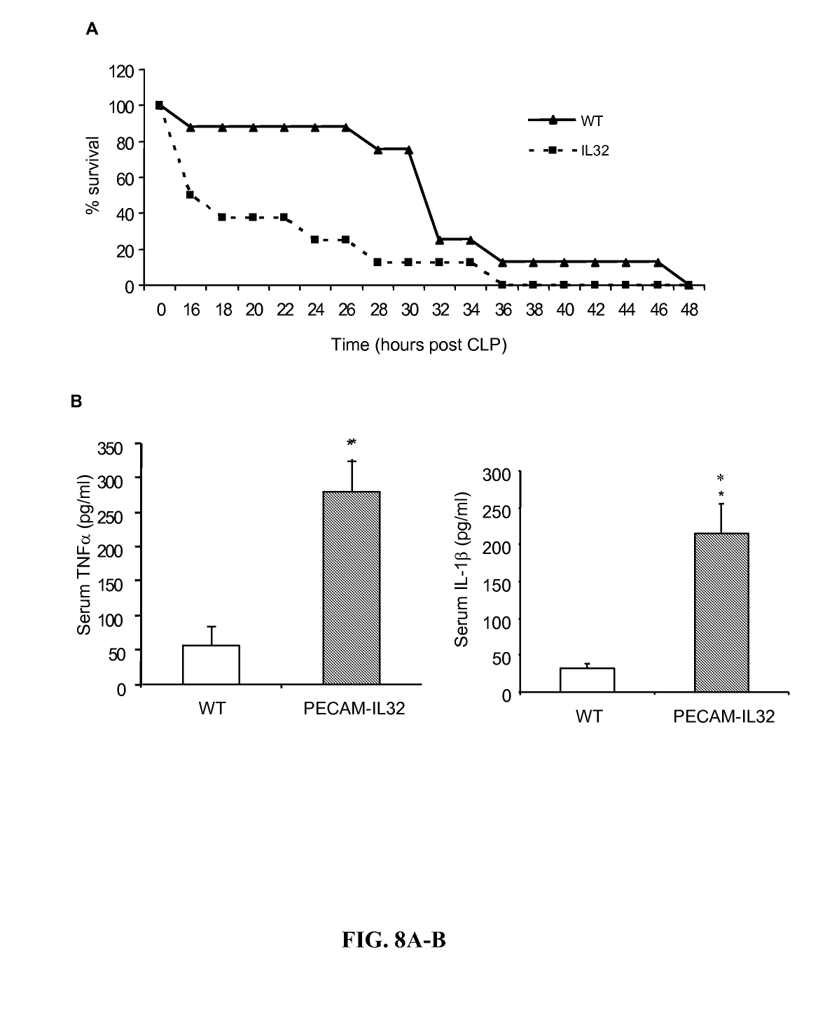

IL-32β promotes inflammation and exacerbates sepsis in vivo. To directly demonstrate the contribution of IL-32β in inflammation in vivo, the inventor has generated a transgenic mouse line (PECAM-IL32), in which IL-32β is driven by an endothelial specific promoter, the PECAM-1 promoter. The mice are groassly healthy, viable and fertile. The inventor isolated endothelial cells from lungs of PECAM-IL32 mice and confirmed transgene expression (FIG. 8A). Accordingly, there is a significant increase in leukocyte adhesion in lung endothelial cells isolated from the transgenic mice compared to wild-type controls when prestimulated with low does of IL-1β (FIG. 8B). The expression of IL-32 in the PECAM-IL32 mice was confirmed in isolated endothelial cells and bone marrow cells (BMCs) using qRT-PCR. IL-32 expression in PECAM-IL32 mice was compared with wild-type which do not have IL-32 gene; 2656-fold higher in BMCs and 133-fold higher in endothelial cells (data not shown).

Sepsis is a systemic inflammatory response to bacterial infection. It occurs when the body's normal reaction to inflammation or a bacterial infection goes into overdrive. A widespread inflammation causes rapid changes in body temperature, blood pressure, vascular permeability and dysfunction in the lung and other organs. It often leads to death. Therefore, the inventor tested whether IL-32β contributes to inflammatory reaction and sepsis development using a cecal-ligation and puncture (CLP) model. Sepsis was induced in ten week-old sex matched wild-type or PECAM-IL32 mice. As expected, animal death was significant accelerated in PECAM-IL32 mice compared to WT control mice (FIG. 8C).

Lung tissues were harvested and analyzed. There is a significant increase in inflammatory cell infiltration into lungs and severe tissue damage in IL-32 transgenic mice compared to the ones from control mice (FIG. 8D). In addition, the inventor examined vascular permeability under sepsis conditions. Six hours after the CLP, he established a vascular window in the back skin fold of the mice, followed by intravenously injection of rhodamine-dextran conjugate. The inventor detected a dramatic increase in vascular leakiness in IL-32 mice compared to controls (FIG. 8E). Blood levels of TNFα and IL-1β were also measured 17 hours following the CLP. Both TNFα and IL-1β level were significantly higher in PECAM-IL32 mice compared to WT mice (FIG. 8D), consistent with the phenotype of sepsis conditions. Together with the in vitro studies, these data reveal a critical and positive role of IL-32β in vascular inflammation. IL-32β amplifies and propagates inflammatory reactions that exacerbate sepsis condition and animal death. This finding has significant implication in inflammatory related diseases in human.

### Example 3

### Cancer

Inflammation is a protective reaction elicited by the host in response to infection, injury, and tissue damage. Infiltration of lymphocytes, macrophages, mast cells and neutrophils is a hallmark of inflammatory defenses and tissue repair reactions. Studies from human samples and animal models clearly show the presence of a variety of infiltrating inflammatory cells in tumor tissues. It is now becoming clear that these infiltrating cells are an indispensable participant in the neoplastic process, fostering proliferation, survival and metastasis. They produce an environment and promote tumor angiogenesis and tumor development (Coussens and Werb, 2002; Balkwill and Coussens, 2004; de Visser et al., 2005). Inflammatory macrophages in tumors generate a number of angiogenic cytokines, and high macrophage counts in mammary tumors and are associated with intense angiogenesis and poor prognosis (Goede et al., 1999). Tumor infiltrated inflammatory cells also contribute factors that promote the formation and enlargement of peritumoral lymphatic vessels, eventually allowing a tumor to metastasize to distant organs (Yu and Rak, 2003). Furthermore, a subsequent free radical release by recruited-leukocytes can damage healthy neighboring cells (Hussain et al., 2003). Finally, the inventor's recent findings demonstrate that tumors manipulate host hematopoiesis and generate a large number of myeloid immune suppressor cells. These immune cells infiltrate into tumors and promotes tumor angiogenesis, in addition to their function in host immune suppression (Yang et al., 2004). It has been estimated that chronic infection and associated inflammation contribute to at least one in four of all cancer cases worldwide (Hussain et al., 2003).

Involvement of inflammation in breast cancer was shown as well as in many other cancers. The major circulating cytokines implicated in breast cancer progression include tumor necrosis factor (TNF)-α, interleukin (IL)-6, and IL-8 (Lewis et al., 1995). IL-8, a pro-inflammatory and pro-angiogenic cytokine, strongly correlates with a number of malignant properties of breast cancer, including angiogenesis, cell invasion and metastasis (Bendre et al., 2002; De Larco et al., 2001). It has been shown to positively correlate with clinical progression of human breast cancer as well, both at early stages and advanced disease (Benoy et al., 2004). A positive correlation of IL-8 with IL-1α and IL-1β in human breast cancer specimens was shown as well (Kurtzman et al., 1999).

Vascular endothelium is an indispensable component of inflammation and tumor progression. Angiogenesis, the formation of new blood vessels, is required for both normal development and disease progression. One of the major differences between physiological and pathological angiogenesis is the presence of inflammation, which commonly accompanies pathological angiogenesis. Tissue injury induces inflammation, a fundamental host defense mechanism. There is growing evidence indicating that inflammation triggers angiogenesis (Coussens and Werb, 2002; Coussens et al., 1999). Conversely, angiogenesis facilitates inflammation by supplying inflammatory cells, cytokines and nutrients. A study of the molecular mechanism of pathological angiogenesis offers great potential in understanding the disease mechanisms as well as the development of therapeutic interventions.

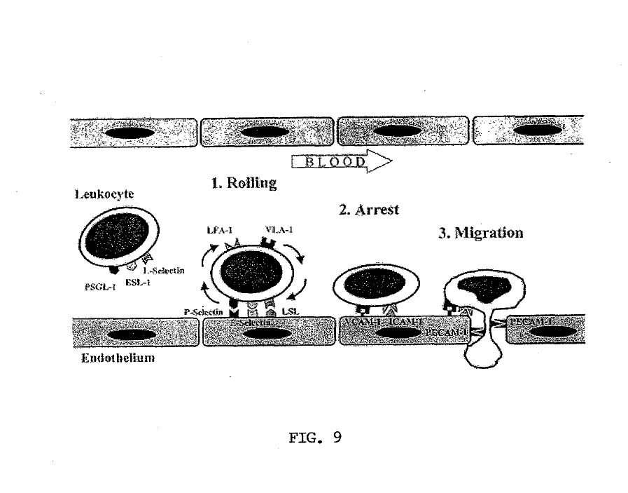

Endothelium is an active participant in the inflammation, and involved in diverse activities including the regulation of leukocyte infiltration, cytokine production, protease and extracellular matrix synthesis, and blood vessel permeability (Folkman, 1995; Folkman, 2001). One of the critical and initial events in inflammation is that migrating leucocytes must first be able to adhere selectively and efficiently to microvascular endothelium in order to withstand the shear force exerted by the flowing blood. Adhesion of leukocyte to endothelium is facilitated by induction of adhesion molecules such as vascular cell adhesion molecule (VCAM)-1, intercellular adhesion molecules (ICAM)-1, and E-selectin and subsequently lead to subendothelial migration (FIG. 9). It is evident that endothelium is a critical gatekeeper that controls the recruitment of distinct leukocyte subpopulations and, in doing so; determines the nature and extent of acute and chronic inflammation. Endothelial cells also exert a potent inflammatory role also by secreting inflammatory cytokines such as IL-1, which upregulates ICAM expression, and stimulates leukocyte adherence to endothelial cell (Krishnaswamy et al., 1999).

IL-32 (a.k.a. NK4). So far, there are only two papers on IL-32. This gene was originally isolated from human activated human NK cells in 1992, and was therefore named NK4. The expression of NK4 is increased after activation of T cells by mitogens or activation of NK cells by IL-2. No homology was found between the sequence of the coding region of this transcript and any sequences in the GenBank data base (Dahl et al., 1992). Very recently (2005), this gene was rediscovered in human lymphocytes upon IL-18 stimulation, and renamed as IL-32 (Kim et al., 2005). Although IL-32 does not share sequence homology with known cytokine families, IL-32 induces various cytokines, human TNFα, and IL-8 in monocytic cells. IL-32 activates NF-κB and p38 mitogen-activated protein kinase in these cells. The IL-32 gene has a full length of 705 bp (accession No. NM 004221) (SEQ ID NO:1 and SEQ ID NO:2). Sequence analysis predicted a molecular weight of 27 kDa. Human IL-32 exists as four splice variants in blood cells, named IL-32α, β, γ and δ, with IL-32α (IL-32) the major isoform in blood cells. The IL-32 peptide contains a RGD motif, indicating that it may be involved in cell-cell adhesion. The highest homology to human IL-32 was found in bovine tissue at 31.8%, and no homologue to this gene was found in mice. Since IL-32 expression is regulated by inflammatory cytokines in human peripheral lymphocyte cells, it has been speculated that it may play a role in inflammatory/autoimmune diseases (Kim et al., 2005).

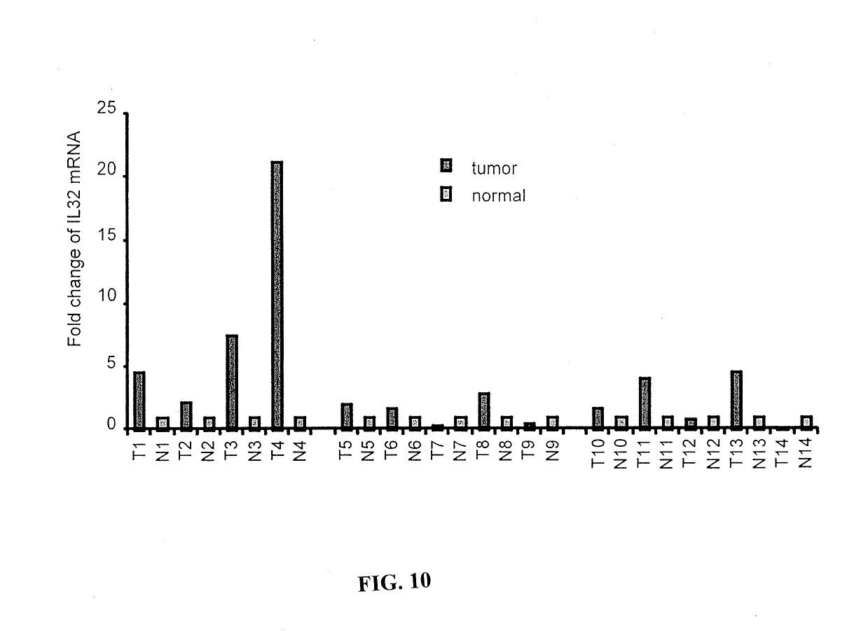

Expression of IL-32β is significantly elevated in breast cancer, GI cancer and brain cancer tissues. It is becoming clear that chronic inflammation promotes tumor development. Thus, the inventor first analyzed IL-32β expression in human breast cancer samples by real time quantitative RT-PCR. The tissue samples were acquired from a large epidemiological study on breast cancer (Lu et al., 2005), in which tumor tissues and adjacent normal breast tissues were collected. In the initial screening of 14 pairs of tissues, the inventor found IL-32β levels significantly increased in 10 tumor samples vs the corresponding normal controls (FIG. 10). In addition, he also examined IL-32β expression. Grade 1, 2 and 4 brain tumors were collected from each patient. Brain tissue sample from epilepsy patients were used as a non-tumor control, since it is very challenge to acquire tissue sample from healthy subject. Total RNA was isolated from the tissue samples and subjected by semi-quantitative RT-PCR using specific primer sets. Consistent with the breast cancer study, the inventor found an increase of IL32β expression in tumor samples compared to normal controls. Most importantly, the inventor observed a correlation of IL-32β levels with brain tumor malignancy. The higher the grade of the tumor, the more IL-32β levels in the tissue (FIG. 11). Collected, these findings suggest IL-32β as a diagnosis marker for cancer detection, as well as a potential target for cancer therapy. It is well documented that anti-inflammatory drugs exhibit potent anti tumor effects, such as drugs targeting the Cox2.

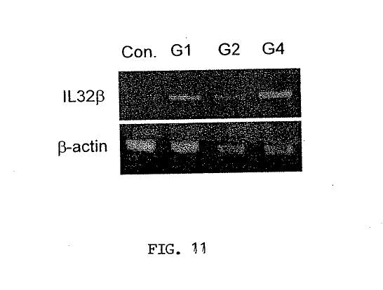

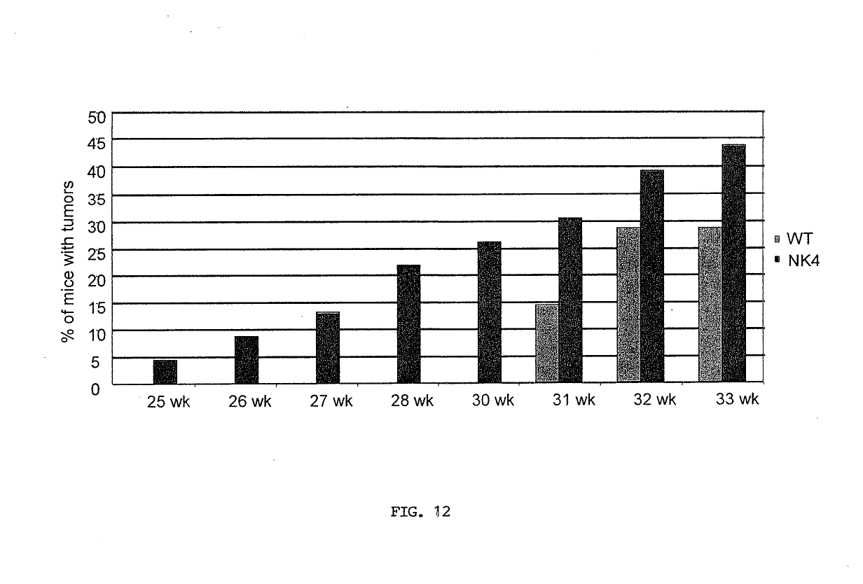

Expression of IL-32β in mice accelerates breast cancer growth and progression. Recent findings demonstrate a critical role of inflammation in tumor development and progression. Based on the data that IL-32β amplifies and propagates inflammation, as well as an elevation of IL-32β in human breast cancers. The inventor reasoned that IL-32β might facilitate tumor growth and progression by modulating inflammation and tumor microenvironment. To test the hypothesis, the inventor have generated a transgenic mouse line in which IL-32β is driven by the PECAM promoter. He used a genetic tumor model, MMTV-neu mice. MMTV-neu mice express the neu antigen in mammary epithelium under the control of the MMTV-LTR promoter and spontaneously develop multifocal mammary adenocarcinoma. The tumor-bearing mice develop metastatic tumors in the lung (Muraoka et al., 2002). This tumor model has a long latency and tumors typically develops when female mice reaches 6-8 months-old. The inventor crossed PECAM-IL-32 mice with MMTV-neu mice. Mammary tumor development in F1PECAM-IL32/MMTV-neu) female mice was monitored starting 3 months after birth. Interestingly, the inventor found an acceleration of tumor growth and progression. Tumor onset occurs at an much early age in the IL-32β transgenic mice compared to wild-type controls (FIG. 12). These findings strongly support a role of IL-32β in tumor development.

### Example 4

### Hair Growth

Recently, the inventor cloned IL-32 from human endothelial cells and found that IL-32 is expressed only in humans and certain closely related primates, but not in rodents and rabbits. Genomic analysis indicates the highest homology to human IL-32 is found in bovine tissue at 31.8%, and no homologue to this gene was found in mice. Four spliced isoforms were cloned. Further analysis indicates that IL-32β is predominantly expressed in vascular endothelial cell, and pro-inflammatory cytokine (TNFα and IL-1β) dramatically upregulates its expression in a NF-κB dependent manner, supporting a role for IL-32β in inflammation. Published results reveal IL-32β induces the degradation of IκBα, the inhibitor for NF-κB, in lymphocytes, illustrating a potential positive feedback regulation of IL-32β in vascular inflammation. Moreover, IL-32β sensitizes endothelial response to IL-1β and TNFα treatment in cell adhesion molecule expression as well as inflammatory cytokine production. As is known, during disease initiation, leukocytes must first adhere to inflamed microvasculature and then transmigrate through the vessel wall before they can accumulate in the target tissues. Thus, the interaction of inflammatory cells with inflamed endothelium is a paramount process in disease development, which includes diabetic wound healing.

Chronic inflammation is a risk factor for wound healing and hair growth. Wound healing is a process that involves both inflammation and the resolution of the inflammatory response, which culminates in remodeling. Uncontrolled or prolonged inflammation often leads to impaired wound healing that is a common complication of diabetes. In fact, a diabetic foot ulcer (DFU) is a leading cause of hospitalization for those patients. In addition, chronic inflammation is also a risk factor for hair loss. Studies have found an inflammatory infiltrate of mononuclear cells and lymphocytes in about 50 percent of the scalp samples observed. Therefore, understanding the molecular mechanisms of diabetic wound healing and hair growth is very significant. Based on the findings of IL-32β in vascular inflammation and inflammation is a risk factor in diabetes, the inventor hypothesized that IL-32β plays a role in diabetic wound healing and hair growth by regulating inflammation.

Interleukin-32 (IL-32) regulates inflammation. IL-32 was first cloned as natural killer cell transcript 4 (NK4) in 1992 and shown to be expressed in IL-2 activated natural killer (NK) cells and T cells (Dahl et al., 1992). Recently, the gene was renamed interleukin-32 (IL-32), due to its ability to induce various cytokines production such as TNFα and IL-8 (Kim et al., 2005). Since IL-32 expression is regulated by inflammatory cytokines in human peripheral lymphocytes, it has been speculated that it may play a role in inflammatory/autoimmune diseases (Kim et al., 2005). Further analysis shows an increase of IL-32 expression in human inflammatory diseases, such as rheumatoid arthritis (Joosten et al., 2006; Cagnard et al., 2005; Shoda et al., 2006) and Crohn's disease (Netea et al., 2005). These clinical findings suggest a role of IL-32 in inflammation and disease progression, which is also supported by the inventor's preliminary data indicating a role of IL-32 in vascular inflammation. Reducing IL-32 activity may benefit patients with inflammation-related diseases including diabetic foot ulcers.

Vascular inflammation is critical in disease progression: Inflammation is a complex biological response of vascular tissues to harmful stimuli. It is a protective attempt by the organism to remove the injurious stimuli as well as to initiate the healing process for the tissue. The vascular endothelium is central to the cellular and molecular events that initiate the response of the body to infection, immune reactions, and tissue injury. Endothelium forms a critical interface between the blood and its components, and damaged tissues (Mori et al., 2002). A variety of stimuli, such as proinflammatory cytokines, injuries, bacterial endotoxins, and viruses, activate endothelium, and lead to alteration of endothelium including loss of vessel barrier function and rapid leukocyte recruitment (Wu et al., 2007). Inflammation that runs unchecked can lead to various diseases, and it is often the underlying cause for many pathological disorders. It is for this reason that inflammation is normally tightly regulated by the body.

The vascular network, being the interface of the two compartments (blood and tissues), plays a key role in inflammation. These inflammatory cells are transported through circulation. During disease initiation, activated/inflamed endothelium is also a major source of inflammatory cytokines that attract circulating leukocytes. Activated endothelium also produces cell adhesion molecules that actively engage in leukocyte adhesion and transendothelial migration, and initiate inflammation. Another important step of the inflammation process is the passage of plasma proteins and leukocytes to the targeted tissues, a process regulated by tight junctions, the adherence junctions, and cell adhesion molecules in blood vessels. The inflammatory cells interact in a sequential multi-step fashion with adhesion molecules on the vascular endothelium; rolling along the endothelial surface by selectins (step 1), firm adhesion by cell adhesion molecules such as intercellular adhesion molecule-1 (ICAM-1), vascular cell adhesion molecule-1 (VCAM-1), and integrins (step 2), and transendothelial migration to the target tissues (step 3) (Omi et al., 2003). As an injury heals, all the events that occurred in the healing process including induced cytokines, recruited inflammatory cells, and activated signaling, go back to normal levels. When that fails, it leads to chronic inflammation. The study and elucidation of endothelial cell dependent mechanisms that regulate leukocyte recruitment is crucial to understanding the inflammatory process. This preliminary data show that IL-32β increases the production of proinflammatory cytokines and adhesion molecules in endothelial cells, indicating a role of IL-32β in vascular inflammation. IL-32β may be a potential therapeutic target to control inflammation in human diseases.

Inflammation is critical in diabetic wound healing. Inflammation is a risk factor in diabetes, and diabetic wound healing is a serious health problem. Inflammation and wound healing are tightly linked and complex processes. Wound healing is a process that involves both inflammation and the resolution of the inflammatory response. Wound healing involves a complex interplay among cells, growth factors, and cytokines. The cascade of events that leads to wound healing begins with clotting and the recruitment of inflammatory cells. The role of macrophages in wound healing-associated inflammation is well documented. Within the first few days after injury, circulating monocytes are recruited into the wound site where they differentiate into macrophages, become activated, phagocytes debris, and produce factors such as vascular endothelial growth factor (VEGF) that regulate angiogenesis and tissue repair. As an injury heals, all the events that occurred in the healing process including induced cytokines, recruited inflammatory cells, and activated signaling, go back to normal levels. Inflammation that runs unchecked can lead to various diseases, and it is often the underlying cause for many pathological disorders, such as diabetes (Loots et al., 1998). It is for this reason that inflammation is normally tightly regulated by the body. In diabetes, limited penetration of blood vessels to the wound will restrict inflammatory cell extravasation at the site, which in turn limits the factors released from these cells. Oxygen and nutrient supply that are required for the activated cells to repair the wound will be limited as well. Besides the effects on angiogenesis, high glucose level is shown to diminish angiogenesis directly in diabetes (Teixeira and Andrade, 1999).

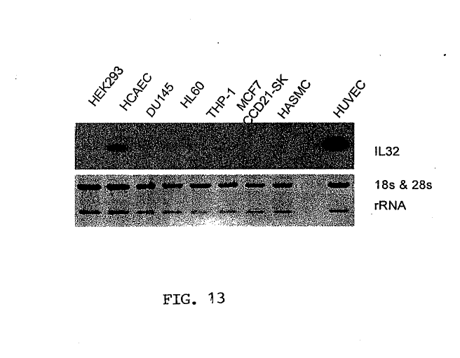

Identification and cloning of human specific IL-32 from human endothelial cells. In searching for novel Akt target genes in endothelial cells, the inventor identified IL-32 that was highly induced by Akt activation. Four splicing isoforms were cloned and the inventor found IL-32β is the predominant isoform in endothelial cells (FIG. 13). Interestingly, the major isoform in monocytes/macrophage is IL-32β (Kim et al., 2005), indicating different isoforms may have different functions in different cells. Further analysis show that IL-32 is expressed in all tested human organs with very low expression in the brain (data not shown).

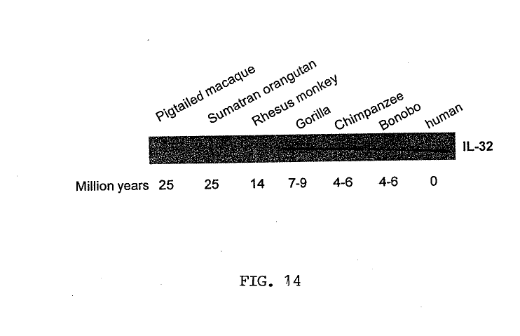

After a blast search against Gene Bank, the inventor noticed that IL-32 does not exist in the published non-human genomes. To verify this, the inventor performed Northern blots and Southern blots for IL-32 against human, mouse, rat and rabbit tissue samples. He confirmed that IL-32 is only present in human samples (data not shown), suggesting IL-32 may be a primate or human specific gene. Therefore, the inventor further analyzed IL-32 expression in primate samples. He found that IL-32 is expressed in certain closely related primates in addition to humans (FIG. 14). This finding supports IL-32 as a human/primate specific gene.

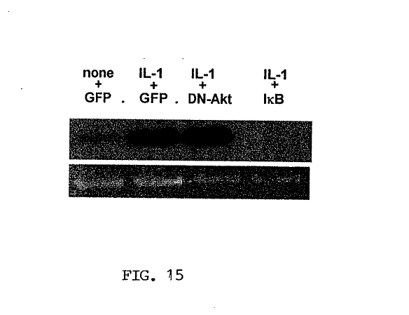

Inflammatory cytokines induce IL-32 expression via NF-κB. The inventor tested a variety of growth factors in cultured human endothelial cells and found that IL-32β is specifically induced by pro-inflammatory cytokines, IL-1β and TNFα. Stimulation of human umbilical vein endothelial cells (HUVECs) with IL-1β induced a robust expression of IL-32 (FIG. 15). Identical data was obtained with TNFα (data not shown). Further analysis showed that proinflammatory cytokine induced IL-32 expression is dependent on NF-κB activity. Neutralization of NF-κB function by overexpressing the inhibitor of NF-κB (IκB) using adenoviral vectors totally abolished proinflammatory cytokine-induced IL-32β expression in endothelial cells (FIG. 15). Interestingly, although overexpression of Akt induced IL-32 expression in endothelial cells that led the inventor to initially clone this gene (data not shown), blocking Akt activation using a dominant negative Akt (DN-Akt) construct has no effects on inflammatory cytokine mediated IL-32 expression (FIG. 15). Additional analysis confirmed that DN-Akt totally blocked Akt activation (data not shown). Collectively, these data suggest that Akt is sufficient but not required in inflammatory cyokine induced IL-32 gene expression.

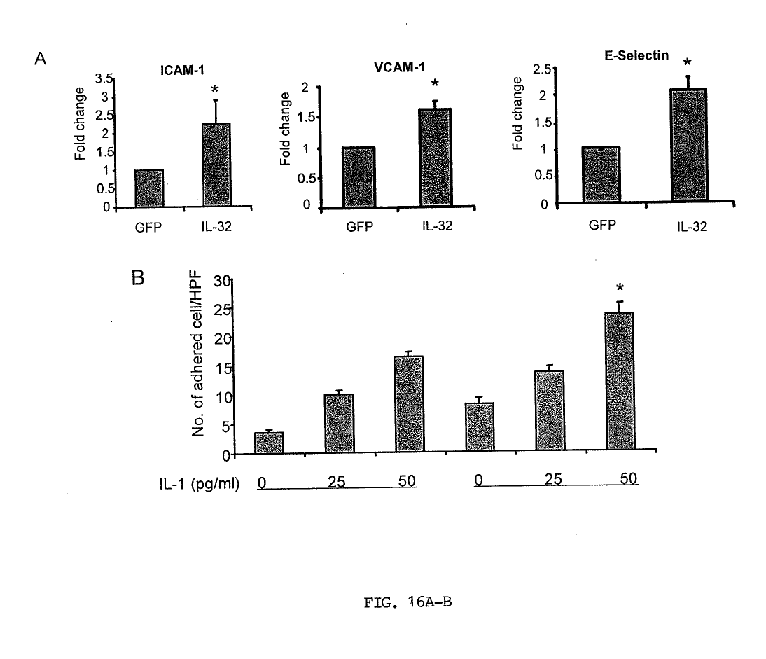

IL-32 is a positive regulator in vascular inflammation. Based on the findings that IL-32 is predominantly expressed in endothelial cells and proinflammatory cytokines regulate its expression, the inventor reasoned that this gene might play a role in vascular inflammation. The expression of cell adhesion molecules, such as ICAM-1, VCAM-1, and E-selectin, on activated endothelium is an initial and critical event in vascular inflammation. Consequently, the inventor investigated the effects of IL-32 in this regard using an adenoviral vector expressing IL-32β (AdIL-32). The inventor found that overexpression of IL-32β alone had no significant effects on the expression of these cell adhesion molecules on endothelial cells (data not shown). However, IL-32β significantly enhanced very low dose of IL-1β stimulation induced expression of ICAM-1, VCAM-1, and E-selectin in HUVECs compared to controls (FIG. 16A). To further evaluate the biological consequences of the elevated levels of cell adhesion molecules in response to IL-32β, the inventor performed a leukocyte adhesion assay using monocytic cells, THP-1. In agreement with the high levels of cell adhesion molecules, there were more THP-1 cells adhered to IL-32β transfected endothelial cells that were treated with a very low dose of IL-1β (FIG. 16B). Collectively, these data demonstrate a contribution of IL-32 to vascular inflammation.

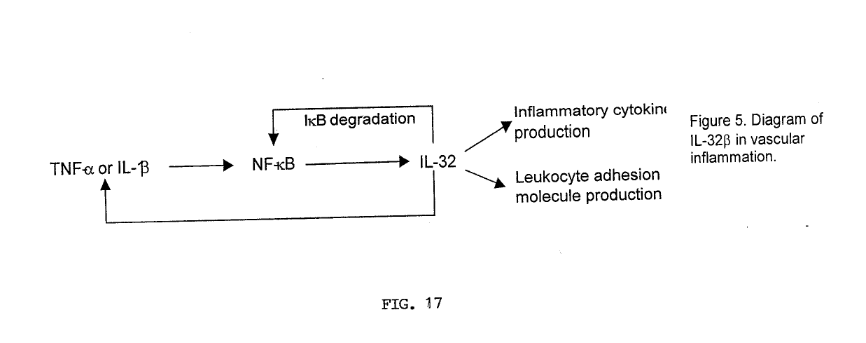

One of the interesting features of proinflammatory cytokines is that they induce their own expression and therefore amplify the inflammatory signal. Since the inventor found that IL-32β sensitizes endothelial cells to inflammatory cytokine stimulation, he examined whether IL-32β also enhances cytokine expression. HUVECs were challenged with IL-1β in combination with IL-32β, followed by measurement of expression of IL-1β. Indeed, the inventor observed an up-regulation of IL-1β upon stimulation with IL-32β in combination with IL-1β compared to either treatment alone in endothelial cells (data not shown). Identical results were observed in TNF-α treated cells (data not shown). Based on published results showing that IL-32 induces IκB degradation in blood cells (Kim et al., 2005) and the inventor's preliminary data, it is proposed that IL-32β functions as a positive mediator in inflammation by modulating the NF-κB activity (FIG. 17). The inventor suggests that IL-32β amplify and prolong the inflammatory reaction in vascular endothelium.

Development of transgenic mice expressing IL-32β in endothelium for the study of vascular inflammation in vivo. Since IL-32 is expressed only in humans and certain primates, there are no conventional animal models that can be used to study its function in vivo. To overcome this limitation, the inventor developed a transgenic mouse line, in which IL-32β gene is driven by an endothelial cell specific promoter, the platelet endothelial cell adhesion molecule-1 (PECAM-1) promoter (PECAM-IL32 mice). The mice are viable, healthy and fertile. The inventor confirmed IL-32β expression in vascular endothelium in PECAM-IL32 mice (data not shown). Although IL-32 is not present in mice, efficacy of exogenous IL-32 in mice has been demonstrated (Joosten et al., 2006; Shoda et al., 2006). The inventor also found enhanced inflammatory reaction by measuring leukocyte infiltration and TNFα production in lungs in PECAM-IL32 mice when challenged with LPS compared to wild-type control mice (data not shown).

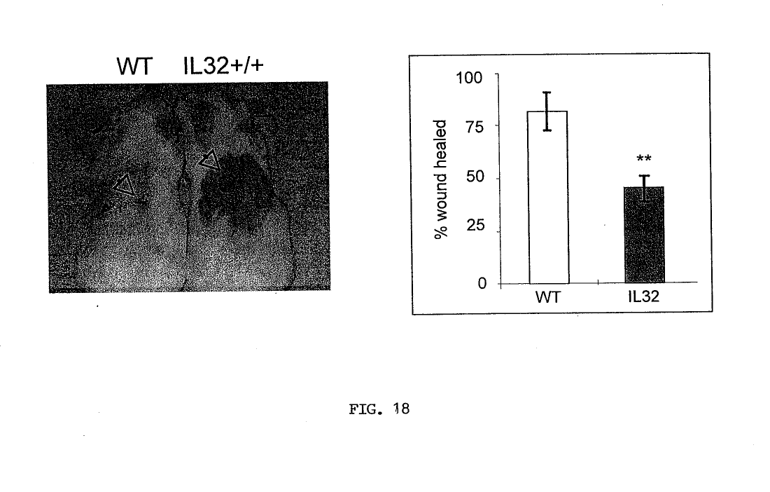

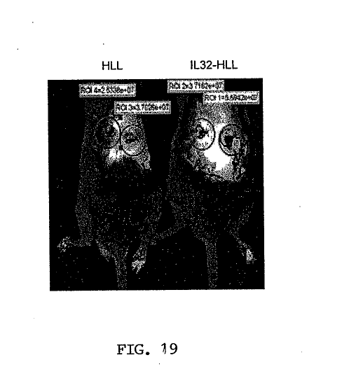

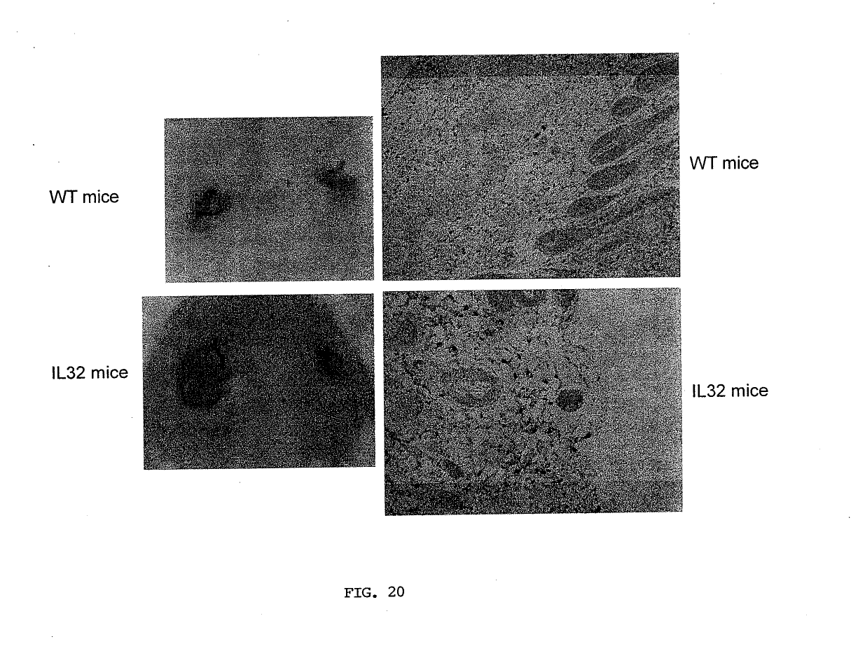

To directly assess the function of IL-32β in wound healing in vivo, excisional wounds were made on PECAM-IL32 mice or wild-type mice (8 wk-old). The inventor observed a significant delay in wound healing in IL-32 mice (FIG. 18) with increased macrophage cell infiltration evaluated by CD68-staining in wounds compared to wild-type (WT) mice (FIG. 20), which is consistent with the inventor's assessment that IL-32β amplifies inflammation that contributes to impaired wound healing. NF-κB transcription factor is a master regulator of inflammation. Therefore, activation of NF-κB could be used as a reporter for inflammation. A transgenic reporter mouse model has been engineered to have an NF-κB responsive promoter driving the expression of luciferase (HLL mice) (Yull, 2003; Sadikot, 2003). The inventor has crossed PECAM-IL32 mice with HLL mice (IL-32-HLL mice) and examined inflammatory reaction during wound healing. The data show a significant increase in luciferase activity, an indicative of inflammation, in IL-32β mice compared to WT controls (FIG. 19), which is in agreement with histological data of increased inflammatory cell infiltration (FIG. 20).

In addition, the inventor observed a dramatic impairment of hair growth in IL-32β transgenic mice. In order to create skin wound, the inventor cut hair with clippers and subsequently removed any residue hair with Nair. Hair regrew in a few days as demonstrated in the wild-type mice. In contrast, hair failed to regrew in the IL-32 transgenic mice (FIG. 20, left panel). Skin tissues around the wound area were harvested and processed for histological analysis. Consistent with the hair growth data, hair follicles in the IL-32 mice display abnormal morphology compared to the one in the wild-type mice that exhibit elongated follicles with growing hair (FIG. 20, right panel). In addition, there are significantly more infiltrating macrophages in the tissues from IL-32β mice than in wild-type controls (FIG. 20, right panel). Collectively, these findings suggest IL-32β enhances inflammation that may lead to impaired hair growth.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference:
U.S. Pat. No. 4,415,732
U.S. Pat. No. 4,458,066
U.S. Pat. No. 4,554,101
U.S. Pat. No. 4,683,195
U.S. Pat. No. 4,683,202
U.S. Pat. No. 4,800,159
U.S. Pat. No. 4,873,191
U.S. Pat. No. 4,883,750
U.S. Pat. No. 4,946,778
U.S. Pat. No. 5,279,721
U.S. Pat. No. 5,354,855
U.S. Pat. No. 5,795,715
U.S. Pat. No. 5,888,773
U.S. Pat. No. 5,889,136
Baker et al., Surgery, 94:331-335, 1983.
Balkwill and Coussens, Nature, 431(7007):405-406, 2004.
Barany and Merrifield, In: The Peptides, Gross and Meienhofer (Eds.), Academic Press, NY, 1-284, 1979.
Bellus, J. Macromol. Sci. Pure Appl. Chem., A31(1): 1355-1376, 1994.
Bendre et al., Cancer Res., 62(19):5571-5579, 2002.
Benoy et al., Clin. Cancer Res., 10(21):7157-7162, 2004.
Bosher and Labouesse, Nat. Cell. Biol., 2(2):E31-E36, 2000.
Brinster et al., Proc. Natl. Acad. Sci. USA, 82(13):4438-4442, 1985.
Cagnard et al., Eur. Cytokine Netw., 16:289-292, 2005.
Capaldi et al., Biochem. Biophys. Res. Comm., 76:425, 1977.
Caplen et al., Gene, 252(1-2):95-105, 2000.
Carter et al., J. Virol., 76(20):10427-10436, 2002.
Cook et al., Cell, 27:487-496, 1981.
Coussens and Werb, Nature, 420(6917):860-867, 2002.
Coussens et al., Genes Dev., 13(11):1382-1397, 1999.
Dahl et al., J. Immunol., 148:597-603, 1992.
De Larco et al., Am. J. Pathol., 158(2):639-646, 2001.
de Visser et al., Cancer Cell, 7(5):411-423, 2005.
DeBusk et al., Exp. Cell Res., 298:167-177, 2004.
Elbashir et al., EMBO, 20(23):6877-6888, 2001.
Elbashir et al., Genes Dev., 5(2):188-200, 2001.
Elbashir et al., Nature, 411(6836):494-498, 2001.
EPO Appln. 320 308
EPO Appln. 329 822
Fire et al., Nature, 391(6669):806-811, 1998.
Foder et al., Science, 251:767-773, 1991.
Folkman, Nat. Med., 1, 1:27-31, 1995.
Folkman, Semin. Oncol., 28:536-542, 2001.
Forster and Symons, Cell, 49:211-220, 1987.
Freifelder, In: Physical Biochemistry Applications to Biochemistry and Molecular Biology, 2nd Ed. Wm. Freeman and Co., NY, 1982.
Frohman, In: PCR Protocols: A Guide To Methods And Applications, Academic Press, N.Y., 1990.
GB Appln. 2 202 328
Gerlach et al., Nature (London), 328:802-805, 1987.
Goda et al., Int. Immunol., 18:233-240, 2006.
Goede et al., Int. J. Cancer, 82(5):765-770, 1999.
Grishok et al., Science, 287:2494-2497, 2000.
Hacia et al., Nature Genet., 14:441-449, 1996.
Hogan et al., In: Manipulating the Mouse Embryo: A Laboratory Manual, 2nd ed., Cold Spring Harbor Laboratory Press, 1994.
Hussain et al., Nat. Rev. Cancer, 3(4):276-285, 2003.
Innis, et al., In: PCR Protocols. A guide to Methods and Application, Academic Press, Inc. San Diego, 1990.
Jobin et al., J. Immunol., 160:410-418, 1998.
Johnson et al., IN: Biotechnology And Pharmacy, Pezzuto et al., (Eds.), Chapman and Hall, New York, 1993.
Joosten et al., Proc. Natl. Acad. Sci. USA, 103:3298-3303, 2006.
Joyce, Nature, 338:217-244, 1989.
Kamiyama et al., Oncogene, 25:7019-7028, 2006.
Ketting et al., Cell, 99(2):133-141, 1999.
Kim and Cech, Proc. Natl. Acad. Sci. USA, 84:8788-8792, 1987.
Kim et al., Immunity, 22:131-142, 2005.
Kotani et al., J. Biol. Chem., 274:21305-21312, 1999.
Krishnaswamy et al., J. Interferon Cytokine Res., 19(2):91-104, 1999.
Kurtzman et al., Oncol. Rep., 6(1):65-70, 1999.
Kwoh et al., Proc. Natl. Acad. Sci. USA, 86(4):1173-1177, 1989.
Kyte and Doolittle, J. Mol. Biol., 157(1):105-132, 1982.
Lewis et al., J. Leukoc. Biol., 57(5):747-751, 1995.
Lin et al., Genetics, 153:1245-1256, 1999.
Lin et al., Proc. Natl. Acad. Sci. USA, 95:8829-8834, 1998.
Loots et al., J. Invest. Dermatol., 111:850-857, 1998.
Lu et al., Cancer Res., 65(12):5015-5019, 2005.
Merrifield, Science, 232(4748):341-347 1986.
Michel and Westhof, J. Mol. Biol., 216:585-610, 1990.
Montgomery et al., Proc. Natl. Acad. Sci. USA, 95:15502-15507, 1998.
Mori et al., FASEB J, 16:963-974, 2002.
Muraoka et al. J. Clin. Invest., 109(12):1551-1559, 2002.
Netea et al., Proc. Natl. Acad. Sci. USA, 102:16309-16314, 2005.
Ohara et al., Proc. Natl. Acad. Sci. USA, 86:5673-5677, 1989.
Omi et al., Microvasc Res., 65:118-124, 2003.
PCT Appln. PCT/US87/00880
PCT Appln. PCT/US89/01025
PCT Appln. WO 00/44914
PCT Appln. WO 01/36646
PCT Appln. WO 01/68836
PCT Appln. WO 88/10315
PCT Appln. WO 89/06700
PCT Appln. WO 90/07641
PCT Appln. WO 99/32619
Pease et al., Proc. Natl. Acad. Sci. USA, 91:5022-5026, 1994.
Pignon J et al., Hum. Mutat., 3(2):126-132, 1994.
Reinhold-Hurek and Shub, Nature, 357:173-176, 1992.
Remick et al., Infect. Immun., 73:2751-2757, 2005.
Remington's Pharmaceutical Sciences, 15^{th} ed., pages 1035-1038 and 1570-1580, Mack Publishing Company, Easton, Pa., 1980.
Remington's Pharmaceutical Sciences 15th Edition, 33:624-652, 1990
Sadikot et al., J. Immunol., 170(2):1091-1098, 2003.
Sambrook et al., In: Molecular Cloning: A Laboratory Manual, Vol. 1, Cold Spring Harbor Laboratory Press, Cold Spring Harbor, N.Y., Ch. 7, 7.19-17.29, 1989.
Sarver et al., Science, 247:1222-1225, 1990.
Scanlon et al., Proc. Natl. Acad. Sci. USA, 88:10591-10595, 1991.
Sharp et al., J. Cereb. Blood Flow Metab., 20:1011-1032, 2000.
Sharp, Genes Dev., 13:139-141, 1999.
Shiojima and Walsh, Circ. Res., 90:1243-1250, 2002.
Shioya et al., Clin. Exp. Immunol., 149:480-486, 2007.
Shoda et al., Arthritis Res. Ther., 8:R166, 2006.
Shoemaker et al., Nature Genetics, 14:450-456, 1996.
Stewart and Young, “Solid Phase Peptide Synthesis”, 2d. ed., Pierce Chemical Co., 1984.
Tabara et al., Cell, 99(2): 123-132, 1999.
Tam et al., J. Am. Chem. Soc., 105:6442, 1983.
Teixeira and Andrade, Life Sci., 64:655-662, 1999.
Terry et al., Transgenic Res., 6:349-356, 1997.
Uehara et al., J. Immunol., 173(6):4179-4189, 2004.
Walker et al., Nucleic Acids Res., 20(7):1691-1696, 1992.
Wincott et al., Nucleic Acids Res., 23(14):2677-2684, 1995.
Wu and Wallace, Genomics, 4:560-569, 1989.
Wu et al., Am. J. Physiology, 293:E1836-1844, 2007.
Yang et al., Am. J. Pathol., 158(2):581-592, 2001.
Yang et al., Cancer Cell, 6(4):409-421, 2004.
Yu and Rak, Breast Cancer Res., 5(2):83-88, 2003.
Yull et al., J. Histochem. Cytochem., 51(6):741-749, 2003.

1. A method of diagnosing cancer comprising:
(a) obtaining a cell sample from a subject suspected of having or at risk of developing cancer;
(b) assessing IL-32β expression in one or more cells of said sample; and
(c) comparing IL-32β expression to that observed in one or more normal cells;
wherein elevated IL-32β expression in the cell or cells of step (b), as compared to the cell or cells of step (c), indicates that said subject has cancer.

2. The method of claim 1, wherein said cancer is breast cancer.

3. The method of claim 1, wherein assessing comprises detection of protein levels.

4. (canceled)

5. The method of claim 1, wherein assessing comprises detection of mRNA levels.

6. (canceled)

7. The method of claim 1, further comprising assessing IL-32β expression in one or more normal cells.

8. The method of claim 1, further comprising making a treatment decision based on the result in step (c).

9. The method of claim 1, wherein said sample is blood or serum.

10. (canceled)

11. A method of treating cancer in a subject, said cancer being characterized by overexpression of IL-32β, comprising administering to said subject an antagonist of IL-32β function or expression.

12. The method of claim 11, wherein said cancer is breast cancer.

13. The method of claim 11, wherein said antagonist is an antibody to IL-32β, a peptide or polypeptide that blocks IL-32β to its receptor, an inhibitory RNA or a small molecule.

14-16. (canceled)

17. The method of claim 11, wherein said antagonist is administered topically, orally, intravenously, intra-arterially, subcutaneously, intradermally, or intratumorally.

18. The method of claim 11, further comprising administering to said subject a second cancer therapy.

19-20. (canceled)

21. A method or promoting or restoring hair growth in a subject comprising administering to a hair-producing cell of said subject an antagonist of IL-32β function or expression.

22-29. (canceled)

30. A method of treating sepsis in a subject comprising administering to said subject an IL-32β-specific antagonist.

31. A method promoting wound healing in a subject comprising administering to said subject an antagonist of IL-32β function or expression.

32-40. (canceled)
